SEQUENCE LISTING (438 sequences)

SEQ ID NO: 1 - protein, 14 aa, synthetic construct
  REGION 1..14: note = linker
GPPPPPPPPPPPPG

SEQ ID NO: 2 - protein, 14 aa, synthetic construct
  REGION 1..14: note = linker
PGPPPPPPPPPPPG

SEQ ID NO: 3 - protein, 14 aa, synthetic construct
  REGION 1..14: note = linker
GGPPPPPPPPPPPG

SEQ ID NO: 4 - protein, 14 aa, synthetic construct
  REGION 1..14: note = linker
PPGPPPPPPPPPPG

SEQ ID NO: 5 - protein, 14 aa, synthetic construct
  REGION 1..14: note = linker
GPGPPPPPPPPPPG

SEQ ID NO: 6 - protein, 14 aa, synthetic construct
  REGION 1..14: note = linker
PGGPPPPPPPPPPG

SEQ ID NO: 7 - protein, 14 aa, synthetic construct
  REGION 1..14: note = linker
GGGPPPPPPPPPPG

SEQ ID NO: 8 - protein, 14 aa, synthetic construct
  REGION 1..14: note = linker
PPPGPPPPPPPPPG

SEQ ID NO: 9 - protein, 14 aa, synthetic construct
  REGION 1..14: note = linker
GPPGPPPPPPPPPG

SEQ ID NO: 10 - protein, 14 aa, synthetic construct
  REGION 1..14: note = linker
PGPGPPPPPPPPPG

SEQ ID NO: 11 - protein, 14 aa, synthetic construct
  REGION 1..14: note = linker
GGPGPPPPPPPPPG

SEQ ID NO: 12 - protein, 14 aa, synthetic construct
  REGION 1..14: note = linker
PPGGPPPPPPPPPG

SEQ ID NO: 13 - protein, 14 aa, synthetic construct
  REGION 1..14: note = linker
GPGGPPPPPPPPPG

SEQ ID NO: 14 - protein, 14 aa, synthetic construct
  REGION 1..14: note = linker
PGGGPPPPPPPPPG

SEQ ID NO: 15 - protein, 14 aa, synthetic construct
  REGION 1..14: note = linker
PPPPGPPPPPPPPG

SEQ ID NO: 16 - protein, 14 aa, synthetic construct
  REGION 1..14: note = linker
GPPPGPPPPPPPPG

SEQ ID NO: 17 - protein, 14 aa, synthetic construct
  REGION 1..14: note = linker
PGPPGPPPPPPPPG

SEQ ID NO: 18 - protein, 14 aa, synthetic construct
  REGION 1..14: note = linker
GGPPGPPPPPPPPG

SEQ ID NO: 19 - protein, 14 aa, synthetic construct
  REGION 1..14: note = linker
PPGPGPPPPPPPPG

SEQ ID NO: 20 - protein, 14 aa, synthetic construct
  REGION 1..14: note = linker
GPGPGPPPPPPPPG

SEQ ID NO: 21 - protein, 14 aa, synthetic construct
  REGION 1..14: note = linker
PGGPGPPPPPPPPG

SEQ ID NO: 22 - protein, 14 aa, synthetic construct
  REGION 1..14: note = linker
PPPGGPPPPPPPPG

SEQ ID NO: 23 - protein, 14 aa, synthetic construct
  REGION 1..14: note = linker
GPPGGPPPPPPPPG

SEQ ID NO: 24 - protein, 14 aa, synthetic construct
  REGION 1..14: note = linker
PGPGGPPPPPPPPG

SEQ ID NO: 25 - protein, 14 aa, synthetic construct
  REGION 1..14: note = linker
PPGGGPPPPPPPPG

SEQ ID NO: 26 - protein, 14 aa, synthetic construct
  REGION 1..14: note = linker
PPPPPGPPPPPPPG

SEQ ID NO: 27 - protein, 14 aa, synthetic construct
  REGION 1..14: note = linker
GPPPPGPPPPPPPG

SEQ ID NO: 28 - protein, 14 aa, synthetic construct
  REGION 1..14: note = linker
PGPPPGPPPPPPPG

SEQ ID NO: 29 - protein, 14 aa, synthetic construct
  REGION 1..14: note = linker
GGPPPGPPPPPPPG

SEQ ID NO: 30 - protein, 14 aa, synthetic construct
  REGION 1..14: note = linker
PPGPPGPPPPPPPG

SEQ ID NO: 31 - protein, 14 aa, synthetic construct
  REGION 1..14: note = linker
GPGPPGPPPPPPPG

SEQ ID NO: 32 - protein, 14 aa, synthetic construct
  REGION 1..14: note = linker
PGGPPGPPPPPPPG

SEQ ID NO: 33 - protein, 14 aa, synthetic construct
  REGION 1..14: note = linker
PPPGPGPPPPPPPG

SEQ ID NO: 34 - protein, 14 aa, synthetic construct
  REGION 1..14: note = linker
GPPGPGPPPPPPPG

SEQ ID NO: 35 - protein, 14 aa, synthetic construct
  REGION 1..14: note = linker
PGPGPGPPPPPPPG

SEQ ID NO: 36 - protein, 14 aa, synthetic construct
  REGION 1..14: note = linker
PPGGPGPPPPPPPG

SEQ ID NO: 37 - protein, 14 aa, synthetic construct
  REGION 1..14: note = linker
PPPPGGPPPPPPPG

SEQ ID NO: 38 - protein, 14 aa, synthetic construct
  REGION 1..14: note = linker
GPPPGGPPPPPPPG

SEQ ID NO: 39 - protein, 14 aa, synthetic construct
  REGION 1..14: note = linker
PGPPGGPPPPPPPG

SEQ ID NO: 40 - protein, 14 aa, synthetic construct
  REGION 1..14: note = linker
PPGPGGPPPPPPPG

SEQ ID NO: 41 - protein, 14 aa, synthetic construct
  REGION 1..14: note = linker
PPPGGGPPPPPPPG

SEQ ID NO: 42 - protein, 14 aa, synthetic construct
  REGION 1..14: note = linker
PPPPPPGPPPPPPG

SEQ ID NO: 43 - protein, 14 aa, synthetic construct
  REGION 1..14: note = linker
GPPPPPGPPPPPPG

SEQ ID NO: 44 - protein, 14 aa, synthetic construct
  REGION 1..14: note = linker
PGPPPPGPPPPPPG

SEQ ID NO: 45 - protein, 14 aa, synthetic construct
  REGION 1..14: note = linker
GGPPPPGPPPPPPG

SEQ ID NO: 46 - protein, 14 aa, synthetic construct
  REGION 1..14: note = linker
PPGPPPGPPPPPPG

SEQ ID NO: 47 - protein, 14 aa, synthetic construct
  REGION 1..14: note = linker
GPGPPPGPPPPPPG

SEQ ID NO: 48 - protein, 14 aa, synthetic construct
  REGION 1..14: note = linker
PGGPPPGPPPPPPG

SEQ ID NO: 49 - protein, 14 aa, synthetic construct
  REGION 1..14: note = linker
PPPGPPGPPPPPPG

SEQ ID NO: 50 - protein, 14 aa, synthetic construct
  REGION 1..14: note = linker
GPPGPPGPPPPPPG

SEQ ID NO: 51 - protein, 14 aa, synthetic construct
  REGION 1..14: note = linker
PGPGPPGPPPPPPG

SEQ ID NO: 52 - protein, 14 aa, synthetic construct
  REGION 1..14: note = linker
PPGGPPGPPPPPPG

SEQ ID NO: 53 - protein, 14 aa, synthetic construct
  REGION 1..14: note = linker
PPPPGPGPPPPPPG

SEQ ID NO: 54 - protein, 14 aa, synthetic construct
  REGION 1..14: note = linker
GPPPGPGPPPPPPG

SEQ ID NO: 55 - protein, 14 aa, synthetic construct
  REGION 1..14: note = linker
PGPPGPGPPPPPPG

SEQ ID NO: 56 - protein, 14 aa, synthetic construct
  REGION 1..14: note = linker
PPGPGPGPPPPPPG

SEQ ID NO: 57 - protein, 14 aa, synthetic construct
  REGION 1..14: note = linker
PPPGGPGPPPPPPG

SEQ ID NO: 58 - protein, 14 aa, synthetic construct
  REGION 1..14: note = linker
PPPPPGGPPPPPPG

SEQ ID NO: 59 - protein, 14 aa, synthetic construct
  REGION 1..14: note = linker
GPPPPGGPPPPPPG

SEQ ID NO: 60 - protein, 14 aa, synthetic construct
  REGION 1..14: note = linker
PGPPPGGPPPPPPG

SEQ ID NO: 61 - protein, 14 aa, synthetic construct
  REGION 1..14: note = linker
PPGPPGGPPPPPPG

SEQ ID NO: 62 - protein, 14 aa, synthetic construct
  REGION 1..14: note = linker
PPPGPGGPPPPPPG

SEQ ID NO: 63 - protein, 14 aa, synthetic construct
  REGION 1..14: note = linker
PPPPGGGPPPPPPG

SEQ ID NO: 64 - protein, 14 aa, synthetic construct
  REGION 1..14: note = linker
PPPPPPPGPPPPPG

SEQ ID NO: 65 - protein, 14 aa, synthetic construct
  REGION 1..14: note = linker
GPPPPPPGPPPPPG

SEQ ID NO: 66 - protein, 14 aa, synthetic construct
  REGION 1..14: note = linker
PGPPPPPGPPPPPG

SEQ ID NO: 67 - protein, 14 aa, synthetic construct
  REGION 1..14: note = linker
GGPPPPPGPPPPPG

SEQ ID NO: 68 - protein, 14 aa, synthetic construct
  REGION 1..14: note = linker
PPGPPPPGPPPPPG

SEQ ID NO: 69 - protein, 14 aa, synthetic construct
  REGION 1..14: note = linker
GPGPPPPGPPPPPG

SEQ ID NO: 70 - protein, 14 aa, synthetic construct
  REGION 1..14: note = linker
PGGPPPPGPPPPPG

SEQ ID NO: 71 - protein, 14 aa, synthetic construct
  REGION 1..14: note = linker
PPPGPPPGPPPPPG

SEQ ID NO: 72 - protein, 14 aa, synthetic construct
  REGION 1..14: note = linker
GPPGPPPGPPPPPG

SEQ ID NO: 73 - protein, 14 aa, synthetic construct
  REGION 1..14: note = linker
PGPGPPPGPPPPPG

SEQ ID NO: 74 - protein, 14 aa, synthetic construct
  REGION 1..14: note = linker
PPGGPPPGPPPPPG

SEQ ID NO: 75 - protein, 14 aa, synthetic construct
  REGION 1..14: note = linker
PPPPGPPGPPPPPG

SEQ ID NO: 76 - protein, 14 aa, synthetic construct
  REGION 1..14: note = linker
GPPPGPPGPPPPPG

SEQ ID NO: 77 - protein, 14 aa, synthetic construct
  REGION 1..14: note = linker
PGPPGPPGPPPPPG

SEQ ID NO: 78 - protein, 14 aa, synthetic construct
  REGION 1..14: note = linker
PPGPGPPGPPPPPG

SEQ ID NO: 79 - protein, 14 aa, synthetic construct
  REGION 1..14: note = linker
PPPGGPPGPPPPPG

SEQ ID NO: 80 - protein, 14 aa, synthetic construct
  REGION 1..14: note = linker
PPPPPGPGPPPPPG

SEQ ID NO: 81 - protein, 14 aa, synthetic construct
  REGION 1..14: note = linker
GPPPPGPGPPPPPG

SEQ ID NO: 82 - protein, 14 aa, synthetic construct
  REGION 1..14: note = linker
PGPPPGPGPPPPPG

SEQ ID NO: 83 - protein, 14 aa, synthetic construct
  REGION 1..14: note = linker
PPGPPGPGPPPPPG

SEQ ID NO: 84 - protein, 14 aa, synthetic construct
  REGION 1..14: note = linker
PPPGPGPGPPPPPG

SEQ ID NO: 85 - protein, 14 aa, synthetic construct
  REGION 1..14: note = linker
PPPPGGPGPPPPPG

SEQ ID NO: 86 - protein, 14 aa, synthetic construct
  REGION 1..14: note = linker
PPPPPPGGPPPPPG

SEQ ID NO: 87 - protein, 14 aa, synthetic construct
  REGION 1..14: note = linker
GPPPPPGGPPPPPG

SEQ ID NO: 88 - protein, 14 aa, synthetic construct
  REGION 1..14: note = linker
PGPPPPGGPPPPPG

SEQ ID NO: 89 - protein, 14 aa, synthetic construct
  REGION 1..14: note = linker
PPGPPPGGPPPPPG

SEQ ID NO: 90 - protein, 14 aa, synthetic construct
  REGION 1..14: note = linker
PPPGPPGGPPPPPG

SEQ ID NO: 91 - protein, 14 aa, synthetic construct
  REGION 1..14: note = linker
PPPPGPGGPPPPPG

SEQ ID NO: 92 - protein, 14 aa, synthetic construct
  REGION 1..14: note = linker
PPPPPGGGPPPPPG

SEQ ID NO: 93 - protein, 14 aa, synthetic construct
  REGION 1..14: note = linker
PPPPPPPPGPPPPG

SEQ ID NO: 94 - protein, 14 aa, synthetic construct
  REGION 1..14: note = linker
GPPPPPPPGPPPPG

SEQ ID NO: 95 - protein, 14 aa, synthetic construct
  REGION 1..14: note = linker
PGPPPPPPGPPPPG

SEQ ID NO: 96 - protein, 14 aa, synthetic construct
  REGION 1..14: note = linker
GGPPPPPPGPPPPG

SEQ ID NO: 97 - protein, 14 aa, synthetic construct
  REGION 1..14: note = linker
PPGPPPPPGPPPPG

SEQ ID NO: 98 - protein, 14 aa, synthetic construct
  REGION 1..14: note = linker
GPGPPPPPGPPPPG

SEQ ID NO: 99 - protein, 14 aa, synthetic construct
  REGION 1..14: note = linker
PGGPPPPPGPPPPG

SEQ ID NO: 100 - protein, 14 aa, synthetic construct
  REGION 1..14: note = linker
PPPGPPPPGPPPPG

SEQ ID NO: 101 - protein, 14 aa, synthetic construct
  REGION 1..14: note = linker
GPPGPPPPGPPPPG

SEQ ID NO: 102 - protein, 14 aa, synthetic construct
  REGION 1..14: note = linker
PGPGPPPPGPPPPG

SEQ ID NO: 103 - protein, 14 aa, synthetic construct
  REGION 1..14: note = linker
PPGGPPPPGPPPPG

SEQ ID NO: 104 - protein, 14 aa, synthetic construct
  REGION 1..14: note = linker
PPPPGPPPGPPPPG

SEQ ID NO: 105 - protein, 14 aa, synthetic construct
  REGION 1..14: note = linker
GPPPGPPPGPPPPG

SEQ ID NO: 106 - protein, 14 aa, synthetic construct
  REGION 1..14: note = linker
PGPPGPPPGPPPPG

SEQ ID NO: 107 - protein, 14 aa, synthetic construct
  REGION 1..14: note = linker
PPGPGPPPGPPPPG

SEQ ID NO: 108 - protein, 14 aa, synthetic construct
  REGION 1..14: note = linker
PPPGGPPPGPPPPG

SEQ ID NO: 109 - protein, 14 aa, synthetic construct
  REGION 1..14: note = linker
PPPPPGPPGPPPPG

SEQ ID NO: 110 - protein, 14 aa, synthetic construct
  REGION 1..14: note = linker
GPPPPGPPGPPPPG

SEQ ID NO: 111 - protein, 14 aa, synthetic construct
  REGION 1..14: note = linker
PGPPPGPPGPPPPG

SEQ ID NO: 112 - protein, 14 aa, synthetic construct
  REGION 1..14: note = linker
PPGPPGPPGPPPPG

SEQ ID NO: 113 - protein, 14 aa, synthetic construct
  REGION 1..14: note = linker
PPPGPGPPGPPPPG

SEQ ID NO: 114 - protein, 14 aa, synthetic construct
  REGION 1..14: note = linker
PPPPGGPPGPPPPG

SEQ ID NO: 115 - protein, 14 aa, synthetic construct
  REGION 1..14: note = linker
PPPPPPGPGPPPPG

SEQ ID NO: 116 - protein, 14 aa, synthetic construct
  REGION 1..14: note = linker
GPPPPPGPGPPPPG

SEQ ID NO: 117 - protein, 14 aa, synthetic construct
  REGION 1..14: note = linker
PGPPPPGPGPPPPG

SEQ ID NO: 118 - protein, 14 aa, synthetic construct
  REGION 1..14: note = linker
PPGPPPGPGPPPPG

SEQ ID NO: 119 - protein, 14 aa, synthetic construct
  REGION 1..14: note = linker
PPPGPPGPGPPPPG

SEQ ID NO: 120 - protein, 14 aa, synthetic construct
  REGION 1..14: note = linker
PPPPGPGPGPPPPG

SEQ ID NO: 121 - protein, 14 aa, synthetic construct
  REGION 1..14: note = linker
PPPPPGGPGPPPPG

SEQ ID NO: 122 - protein, 14 aa, synthetic construct
  REGION 1..14: note = linker
PPPPPPPGGPPPPG

SEQ ID NO: 123 - protein, 14 aa, synthetic construct
  REGION 1..14: note = linker
GPPPPPPGGPPPPG

SEQ ID NO: 124 - protein, 14 aa, synthetic construct
  REGION 1..14: note = linker
PGPPPPPGGPPPPG

SEQ ID NO: 125 - protein, 14 aa, synthetic construct
  REGION 1..14: note = linker
PPGPPPPGGPPPPG

SEQ ID NO: 126 - protein, 14 aa, synthetic construct
  REGION 1..14: note = linker
PPPGPPPGGPPPPG

SEQ ID NO: 127 - protein, 14 aa, synthetic construct
  REGION 1..14: note = linker
PPPPGPPGGPPPPG

SEQ ID NO: 128 - protein, 14 aa, synthetic construct
  REGION 1..14: note = linker
PPPPPGPGGPPPPG

SEQ ID NO: 129 - protein, 14 aa, synthetic construct
  REGION 1..14: note = linker
PPPPPPGGGPPPPG

SEQ ID NO: 130 - protein, 14 aa, synthetic construct
  REGION 1..14: note = linker
PPPPPPPPPGPPPG

SEQ ID NO: 131 - protein, 14 aa, synthetic construct
  REGION 1..14: note = linker
GPPPPPPPPGPPPG

SEQ ID NO: 132 - protein, 14 aa, synthetic construct
  REGION 1..14: note = linker
PGPPPPPPPGPPPG

SEQ ID NO: 133 - protein, 14 aa, synthetic construct
  REGION 1..14: note = linker
GGPPPPPPPGPPPG

SEQ ID NO: 134 - protein, 14 aa, synthetic construct
  REGION 1..14: note = linker
PPGPPPPPPGPPPG

SEQ ID NO: 135 - protein, 14 aa, synthetic construct
  REGION 1..14: note = linker
GPGPPPPPPGPPPG

SEQ ID NO: 136 - protein, 14 aa, synthetic construct
  REGION 1..14: note = linker
PGGPPPPPPGPPPG

SEQ ID NO: 137 - protein, 14 aa, synthetic construct
  REGION 1..14: note = linker
PPPGPPPPPGPPPG

SEQ ID NO: 138 - protein, 14 aa, synthetic construct
  REGION 1..14: note = linker
GPPGPPPPPGPPPG

SEQ ID NO: 139 - protein, 14 aa, synthetic construct
  REGION 1..14: note = linker
PGPGPPPPPGPPPG

SEQ ID NO: 140 - protein, 14 aa, synthetic construct
  REGION 1..14: note = linker
PPGGPPPPPGPPPG

SEQ ID NO: 141 - protein, 14 aa, synthetic construct
  REGION 1..14: note = linker
PPPPGPPPPGPPPG

SEQ ID NO: 142 - protein, 14 aa, synthetic construct
  REGION 1..14: note = linker
GPPPGPPPPGPPPG

SEQ ID NO: 143 - protein, 14 aa, synthetic construct
  REGION 1..14: note = linker
PGPPGPPPPGPPPG

SEQ ID NO: 144 - protein, 14 aa, synthetic construct
  REGION 1..14: note = linker
PPGPGPPPPGPPPG

SEQ ID NO: 145 - protein, 14 aa, synthetic construct
  REGION 1..14: note = linker
PPPGGPPPPGPPPG

SEQ ID NO: 146 - protein, 14 aa, synthetic construct
  REGION 1..14: note = linker
PPPPPGPPPGPPPG

SEQ ID NO: 147 - protein, 14 aa, synthetic construct
  REGION 1..14: note = linker
GPPPPGPPPGPPPG

SEQ ID NO: 148 - protein, 14 aa, synthetic construct
  REGION 1..14: note = linker
PGPPPGPPPGPPPG

SEQ ID NO: 149 - protein, 14 aa, synthetic construct
  REGION 1..14: note = linker
PPGPPGPPPGPPPG

SEQ ID NO: 150 - protein, 14 aa, synthetic construct
  REGION 1..14: note = linker
PPPGPGPPPGPPPG

SEQ ID NO: 151 - protein, 14 aa, synthetic construct
  REGION 1..14: note = linker
PPPPGGPPPGPPPG

SEQ ID NO: 152 - protein, 14 aa, synthetic construct
  REGION 1..14: note = linker
PPPPPPGPPGPPPG

SEQ ID NO: 153 - protein, 14 aa, synthetic construct
  REGION 1..14: note = linker
GPPPPPGPPGPPPG

SEQ ID NO: 154 - protein, 14 aa, synthetic construct
  REGION 1..14: note = linker
PGPPPPGPPGPPPG

SEQ ID NO: 155 - protein, 14 aa, synthetic construct
  REGION 1..14: note = linker
PPGPPPGPPGPPPG

SEQ ID NO: 156 - protein, 14 aa, synthetic construct
  REGION 1..14: note = linker
PPPGPPGPPGPPPG

SEQ ID NO: 157 - protein, 14 aa, synthetic construct
  REGION 1..14: note = linker
PPPPGPGPPGPPPG

SEQ ID NO: 158 - protein, 14 aa, synthetic construct
  REGION 1..14: note = linker
PPPPPGGPPGPPPG

SEQ ID NO: 159 - protein, 14 aa, synthetic construct
  REGION 1..14: note = linker
PPPPPPPGPGPPPG

SEQ ID NO: 160 - protein, 14 aa, synthetic construct
  REGION 1..14: note = linker
GPPPPPPGPGPPPG

SEQ ID NO: 161 - protein, 14 aa, synthetic construct
  REGION 1..14: note = linker
PGPPPPPGPGPPPG

SEQ ID NO: 162 - protein, 14 aa, synthetic construct
  REGION 1..14: note = linker
PPGPPPPGPGPPPG

SEQ ID NO: 163 - protein, 14 aa, synthetic construct
  REGION 1..14: note = linker
PPPGPPPGPGPPPG

SEQ ID NO: 164 - protein, 14 aa, synthetic construct
  REGION 1..14: note = linker
PPPPGPPGPGPPPG

SEQ ID NO: 165 - protein, 14 aa, synthetic construct
  REGION 1..14: note = linker
PPPPPGPGPGPPPG

SEQ ID NO: 166 - protein, 14 aa, synthetic construct
  REGION 1..14: note = linker
PPPPPPGGPGPPPG

SEQ ID NO: 167 - protein, 14 aa, synthetic construct
  REGION 1..14: note = linker
PPPPPPPPGGPPPG

SEQ ID NO: 168 - protein, 14 aa, synthetic construct
  REGION 1..14: note = linker
GPPPPPPPGGPPPG

SEQ ID NO: 169 - protein, 14 aa, synthetic construct
  REGION 1..14: note = linker
PGPPPPPPGGPPPG

SEQ ID NO: 170 - protein, 14 aa, synthetic construct
  REGION 1..14: note = linker
PPGPPPPPGGPPPG

SEQ ID NO: 171 - protein, 14 aa, synthetic construct
  REGION 1..14: note = linker
PPPGPPPPGGPPPG

SEQ ID NO: 172 - protein, 14 aa, synthetic construct
  REGION 1..14: note = linker
PPPPGPPPGGPPPG

SEQ ID NO: 173 - protein, 14 aa, synthetic construct
  REGION 1..14: note = linker
PPPPPGPPGGPPPG

SEQ ID NO: 174 - protein, 14 aa, synthetic construct
  REGION 1..14: note = linker
PPPPPPGPGGPPPG

SEQ ID NO: 175 - protein, 14 aa, synthetic construct
  REGION 1..14: note = linker
PPPPPPPGGGPPPG

SEQ ID NO: 176 - protein, 14 aa, synthetic construct
  REGION 1..14: note = linker
PPPPPPPPPPGPPG

SEQ ID NO: 177 - protein, 14 aa, synthetic construct
  REGION 1..14: note = linker
GPPPPPPPPPGPPG

SEQ ID NO: 178 - protein, 14 aa, synthetic construct
  REGION 1..14: note = linker
PGPPPPPPPPGPPG

SEQ ID NO: 179 - protein, 14 aa, synthetic construct
  REGION 1..14: note = linker
GGPPPPPPPPGPPG

SEQ ID NO: 180 - protein, 14 aa, synthetic construct
  REGION 1..14: note = linker
PPGPPPPPPPGPPG

SEQ ID NO: 181 - protein, 14 aa, synthetic construct
  REGION 1..14: note = linker
GPGPPPPPPPGPPG

SEQ ID NO: 182 - protein, 14 aa, synthetic construct
  REGION 1..14: note = linker
PGGPPPPPPPGPPG

SEQ ID NO: 183 - protein, 14 aa, synthetic construct
  REGION 1..14: note = linker
PPPGPPPPPPGPPG

SEQ ID NO: 184 - protein, 14 aa, synthetic construct
  REGION 1..14: note = linker
GPPGPPPPPPGPPG

SEQ ID NO: 185 - protein, 14 aa, synthetic construct
  REGION 1..14: note = linker
PGPGPPPPPPGPPG

SEQ ID NO: 186 - protein, 14 aa, synthetic construct
  REGION 1..14: note = linker
PPGGPPPPPPGPPG

SEQ ID NO: 187 - protein, 14 aa, synthetic construct
  REGION 1..14: note = linker
PPPPGPPPPPGPPG

SEQ ID NO: 188 - protein, 14 aa, synthetic construct
  REGION 1..14: note = linker
GPPPGPPPPPGPPG

SEQ ID NO: 189 - protein, 14 aa, synthetic construct
  REGION 1..14: note = linker
PGPPGPPPPPGPPG

SEQ ID NO: 190 - protein, 14 aa, synthetic construct
  REGION 1..14: note = linker
PPGPGPPPPPGPPG

SEQ ID NO: 191 - protein, 14 aa, synthetic construct
  REGION 1..14: note = linker
PPPGGPPPPPGPPG

SEQ ID NO: 192 - protein, 14 aa, synthetic construct
  REGION 1..14: note = linker
PPPPPGPPPPGPPG

SEQ ID NO: 193 - protein, 14 aa, synthetic construct
  REGION 1..14: note = linker
GPPPPGPPPPGPPG

SEQ ID NO: 194 - protein, 14 aa, synthetic construct
  REGION 1..14: note = linker
PGPPPGPPPPGPPG

SEQ ID NO: 195 - protein, 14 aa, synthetic construct
  REGION 1..14: note = linker
PPGPPGPPPPGPPG

SEQ ID NO: 196 - protein, 14 aa, synthetic construct
  REGION 1..14: note = linker
PPPGPGPPPPGPPG

SEQ ID NO: 197 - protein, 14 aa, synthetic construct
  REGION 1..14: note = linker
PPPPGGPPPPGPPG

SEQ ID NO: 198 - protein, 14 aa, synthetic construct
  REGION 1..14: note = linker
PPPPPPGPPPGPPG

SEQ ID NO: 199 - protein, 14 aa, synthetic construct
  REGION 1..14: note = linker
GPPPPPGPPPGPPG

SEQ ID NO: 200 - protein, 14 aa, synthetic construct
  REGION 1..14: note = linker
PGPPPPGPPPGPPG

SEQ ID NO: 201 - protein, 14 aa, synthetic construct
  REGION 1..14: note = linker
PPGPPPGPPPGPPG

SEQ ID NO: 202 - protein, 14 aa, synthetic construct
  REGION 1..14: note = linker
PPPGPPGPPPGPPG

SEQ ID NO: 203 - protein, 14 aa, synthetic construct
  REGION 1..14: note = linker
PPPPGPGPPPGPPG

SEQ ID NO: 204 - protein, 14 aa, synthetic construct
  REGION 1..14: note = linker
PPPPPGGPPPGPPG

SEQ ID NO: 205 - protein, 14 aa, synthetic construct
  REGION 1..14: note = linker
PPPPPPPGPPGPPG

SEQ ID NO: 206 - protein, 14 aa, synthetic construct
  REGION 1..14: note = linker
GPPPPPPGPPGPPG

SEQ ID NO: 207 - protein, 14 aa, synthetic construct
  REGION 1..14: note = linker
PGPPPPPGPPGPPG

SEQ ID NO: 208 - protein, 14 aa, synthetic construct
  REGION 1..14: note = linker
PPGPPPPGPPGPPG

SEQ ID NO: 209 - protein, 14 aa, synthetic construct
  REGION 1..14: note = linker
PPPGPPPGPPGPPG

SEQ ID NO: 210 - protein, 14 aa, synthetic construct
  REGION 1..14: note = linker
PPPPGPPGPPGPPG

SEQ ID NO: 211 - protein, 14 aa, synthetic construct
  REGION 1..14: note = linker
PPPPPGPGPPGPPG

SEQ ID NO: 212 - protein, 14 aa, synthetic construct
  REGION 1..14: note = linker
PPPPPPGGPPGPPG

SEQ ID NO: 213 - protein, 14 aa, synthetic construct
  REGION 1..14: note = linker
PPPPPPPPGPGPPG

SEQ ID NO: 214 - protein, 14 aa, synthetic construct
  REGION 1..14: note = linker
GPPPPPPPGPGPPG

SEQ ID NO: 215 - protein, 14 aa, synthetic construct
  REGION 1..14: note = linker
PGPPPPPPGPGPPG

SEQ ID NO: 216 - protein, 14 aa, synthetic construct
  REGION 1..14: note = linker
PPGPPPPPGPGPPG

SEQ ID NO: 217 - protein, 14 aa, synthetic construct
  REGION 1..14: note = linker
PPPGPPPPGPGPPG

SEQ ID NO: 218 - protein, 14 aa, synthetic construct
  REGION 1..14: note = linker
PPPPGPPPGPGPPG

SEQ ID NO: 219 - protein, 14 aa, synthetic construct
  REGION 1..14: note = linker
PPPPPGPPGPGPPG

SEQ ID NO: 220 - protein, 14 aa, synthetic construct
  REGION 1..14: note = linker
PPPPPPGPGPGPPG

SEQ ID NO: 221 - protein, 14 aa, synthetic construct
  REGION 1..14: note = linker
PPPPPPPGGPGPPG

SEQ ID NO: 222 - protein, 14 aa, synthetic construct
  REGION 1..14: note = linker
PPPPPPPPPGGPPG

SEQ ID NO: 223 - protein, 14 aa, synthetic construct
  REGION 1..14: note = linker
GPPPPPPPPGGPPG

SEQ ID NO: 224 - protein, 14 aa, synthetic construct
  REGION 1..14: note = linker
PGPPPPPPPGGPPG

SEQ ID NO: 225 - protein, 14 aa, synthetic construct
  REGION 1..14: note = linker
PPGPPPPPPGGPPG

SEQ ID NO: 226 - protein, 14 aa, synthetic construct
  REGION 1..14: note = linker
PPPGPPPPPGGPPG

SEQ ID NO: 227 - protein, 14 aa, synthetic construct
  REGION 1..14: note = linker
PPPPGPPPPGGPPG

SEQ ID NO: 228 - protein, 14 aa, synthetic construct
  REGION 1..14: note = linker
PPPPPGPPPGGPPG

SEQ ID NO: 229 - protein, 14 aa, synthetic construct
  REGION 1..14: note = linker
PPPPPPGPPGGPPG

SEQ ID NO: 230 - protein, 14 aa, synthetic construct
  REGION 1..14: note = linker
PPPPPPPGPGGPPG

SEQ ID NO: 231 - protein, 14 aa, synthetic construct
  REGION 1..14: note = linker
PPPPPPPPGGGPPG

SEQ ID NO: 232 - protein, 14 aa, synthetic construct
  REGION 1..14: note = linker
PPPPPPPPPPPGPG

SEQ ID NO: 233 - protein, 14 aa, synthetic construct
  REGION 1..14: note = linker
GPPPPPPPPPPGPG

SEQ ID NO: 234 - protein, 14 aa, synthetic construct
  REGION 1..14: note = linker
PGPPPPPPPPPGPG

SEQ ID NO: 235 - protein, 14 aa, synthetic construct
  REGION 1..14: note = linker
GGPPPPPPPPPGPG

SEQ ID NO: 236 - protein, 14 aa, synthetic construct
  REGION 1..14: note = linker
PPGPPPPPPPPGPG

SEQ ID NO: 237 - protein, 14 aa, synthetic construct
  REGION 1..14: note = linker
GPGPPPPPPPPGPG

SEQ ID NO: 238 - protein, 14 aa, synthetic construct
  REGION 1..14: note = linker
PGGPPPPPPPPGPG

SEQ ID NO: 239 - protein, 14 aa, synthetic construct
  REGION 1..14: note = linker
PPPGPPPPPPPGPG

SEQ ID NO: 240 - protein, 14 aa, synthetic construct
  REGION 1..14: note = linker
GPPGPPPPPPPGPG

SEQ ID NO: 241 - protein, 14 aa, synthetic construct
  REGION 1..14: note = linker
PGPGPPPPPPPGPG

SEQ ID NO: 242 - protein, 14 aa, synthetic construct
  REGION 1..14: note = linker
PPGGPPPPPPPGPG

SEQ ID NO: 243 - protein, 14 aa, synthetic construct
  REGION 1..14: note = linker
PPPPGPPPPPPGPG

SEQ ID NO: 244 - protein, 14 aa, synthetic construct
  REGION 1..14: note = linker
GPPPGPPPPPPGPG

SEQ ID NO: 245 - protein, 14 aa, synthetic construct
  REGION 1..14: note = linker
PGPPGPPPPPPGPG

SEQ ID NO: 246 - protein, 14 aa, synthetic construct
  REGION 1..14: note = linker
PPGPGPPPPPPGPG

SEQ ID NO: 247 - protein, 14 aa, synthetic construct
  REGION 1..14: note = linker
PPPGGPPPPPPGPG

SEQ ID NO: 248 - protein, 14 aa, synthetic construct
  REGION 1..14: note = linker
PPPPPGPPPPPGPG

SEQ ID NO: 249 - protein, 14 aa, synthetic construct
  REGION 1..14: note = linker
GPPPPGPPPPPGPG

SEQ ID NO: 250 - protein, 14 aa, synthetic construct
  REGION 1..14: note = linker
PGPPPGPPPPPGPG

SEQ ID NO: 251 - protein, 14 aa, synthetic construct
  REGION 1..14: note = linker
PPGPPGPPPPPGPG

SEQ ID NO: 252 - protein, 14 aa, synthetic construct
  REGION 1..14: note = linker
PPPGPGPPPPPGPG

SEQ ID NO: 253 - protein, 14 aa, synthetic construct
  REGION 1..14: note = linker
PPPPGGPPPPPGPG

SEQ ID NO: 254 - protein, 14 aa, synthetic construct
  REGION 1..14: note = linker
PPPPPPGPPPPGPG

SEQ ID NO: 255 - protein, 14 aa, synthetic construct
  REGION 1..14: note = linker
GPPPPPGPPPPGPG

SEQ ID NO: 256 - protein, 14 aa, synthetic construct
  REGION 1..14: note = linker
PGPPPPGPPPPGPG

SEQ ID NO: 257 - protein, 14 aa, synthetic construct
  REGION 1..14: note = linker
PPGPPPGPPPPGPG

SEQ ID NO: 258 - protein, 14 aa, synthetic construct
  REGION 1..14: note = linker
PPPGPPGPPPPGPG

SEQ ID NO: 259 - protein, 14 aa, synthetic construct
  REGION 1..14: note = linker
PPPPGPGPPPPGPG

SEQ ID NO: 260 - protein, 14 aa, synthetic construct
  REGION 1..14: note = linker
PPPPPGGPPPPGPG

SEQ ID NO: 261 - protein, 14 aa, synthetic construct
  REGION 1..14: note = linker
PPPPPPPGPPPGPG

SEQ ID NO: 262 - protein, 14 aa, synthetic construct
  REGION 1..14: note = linker
GPPPPPPGPPPGPG

SEQ ID NO: 263 - protein, 14 aa, synthetic construct
  REGION 1..14: note = linker
PGPPPPPGPPPGPG

SEQ ID NO: 264 - protein, 14 aa, synthetic construct
  REGION 1..14: note = linker
PPGPPPPGPPPGPG

SEQ ID NO: 265 - protein, 14 aa, synthetic construct
  REGION 1..14: note = linker
PPPGPPPGPPPGPG

SEQ ID NO: 266 - protein, 14 aa, synthetic construct
  REGION 1..14: note = linker
PPPPGPPGPPPGPG

SEQ ID NO: 267 - protein, 14 aa, synthetic construct
  REGION 1..14: note = linker
PPPPPGPGPPPGPG

SEQ ID NO: 268 - protein, 14 aa, synthetic construct
  REGION 1..14: note = linker
PPPPPPGGPPPGPG

SEQ ID NO: 269 - protein, 14 aa, synthetic construct
  REGION 1..14: note = linker
PPPPPPPPGPPGPG

SEQ ID NO: 270 - protein, 14 aa, synthetic construct
  REGION 1..14: note = linker
GPPPPPPPGPPGPG

SEQ ID NO: 271 - protein, 14 aa, synthetic construct
  REGION 1..14: note = linker
PGPPPPPPGPPGPG

SEQ ID NO: 272 - protein, 14 aa, synthetic construct
  REGION 1..14: note = linker
PPGPPPPPGPPGPG

SEQ ID NO: 273 - protein, 14 aa, synthetic construct
  REGION 1..14: note = linker
PPPGPPPPGPPGPG

SEQ ID NO: 274 - protein, 14 aa, synthetic construct
  REGION 1..14: note = linker
PPPPGPPPGPPGPG

SEQ ID NO: 275 - protein, 14 aa, synthetic construct
  REGION 1..14: note = linker
PPPPPGPPGPPGPG

SEQ ID NO: 276 - protein, 14 aa, synthetic construct
  REGION 1..14: note = linker
PPPPPPGPGPPGPG

SEQ ID NO: 277 - protein, 14 aa, synthetic construct
  REGION 1..14: note = linker
PPPPPPPGGPPGPG

SEQ ID NO: 278 - protein, 14 aa, synthetic construct
  REGION 1..14: note = linker
PPPPPPPPPGPGPG

SEQ ID NO: 279 - protein, 14 aa, synthetic construct
  REGION 1..14: note = linker
GPPPPPPPPGPGPG

SEQ ID NO: 280 - protein, 14 aa, synthetic construct
  REGION 1..14: note = linker
PGPPPPPPPGPGPG

SEQ ID NO: 281 - protein, 14 aa, synthetic construct
  REGION 1..14: note = linker
PPGPPPPPPGPGPG

SEQ ID NO: 282 - protein, 14 aa, synthetic construct
  REGION 1..14: note = linker
PPPGPPPPPGPGPG

SEQ ID NO: 283 - protein, 14 aa, synthetic construct
  REGION 1..14: note = linker
PPPPGPPPPGPGPG

SEQ ID NO: 284 - protein, 14 aa, synthetic construct
  REGION 1..14: note = linker
PPPPPGPPPGPGPG

SEQ ID NO: 285 - protein, 14 aa, synthetic construct
  REGION 1..14: note = linker
PPPPPPGPPGPGPG

SEQ ID NO: 286 - protein, 14 aa, synthetic construct
  REGION 1..14: note = linker
PPPPPPPGPGPGPG

SEQ ID NO: 287 - protein, 14 aa, synthetic construct
  REGION 1..14: note = linker
PPPPPPPPGGPGPG

SEQ ID NO: 288 - protein, 14 aa, synthetic construct
  REGION 1..14: note = linker
PPPPPPPPPPGGPG

SEQ ID NO: 289 - protein, 14 aa, synthetic construct
  REGION 1..14: note = linker
GPPPPPPPPPGGPG

SEQ ID NO: 290 - protein, 14 aa, synthetic construct
  REGION 1..14: note = linker
PGPPPPPPPPGGPG

SEQ ID NO: 291 - protein, 14 aa, synthetic construct
  REGION 1..14: note = linker
PPGPPPPPPPGGPG

SEQ ID NO: 292 - protein, 14 aa, synthetic construct
  REGION 1..14: note = linker
PPPGPPPPPPGGPG

SEQ ID NO: 293 - protein, 14 aa, synthetic construct
  REGION 1..14: note = linker
PPPPGPPPPPGGPG

SEQ ID NO: 294 - protein, 14 aa, synthetic construct
  REGION 1..14: note = linker
PPPPPGPPPPGGPG

SEQ ID NO: 295 - protein, 14 aa, synthetic construct
  REGION 1..14: note = linker
PPPPPPGPPPGGPG

SEQ ID NO: 296 - protein, 14 aa, synthetic construct
  REGION 1..14: note = linker
PPPPPPPGPPGGPG

SEQ ID NO: 297 - protein, 14 aa, synthetic construct
  REGION 1..14: note = linker
PPPPPPPPGPGGPG

SEQ ID NO: 298 - protein, 14 aa, synthetic construct
  REGION 1..14: note = linker
PPPPPPPPPGGGPG

SEQ ID NO: 299 - protein, 14 aa, synthetic construct
  REGION 1..14: note = linker
PPPPPPPPPPPPGG

SEQ ID NO: 300 - protein, 14 aa, synthetic construct
  REGION 1..14: note = linker
GPPPPPPPPPPPGG

SEQ ID NO: 301 - protein, 14 aa, synthetic construct
  REGION 1..14: note = linker
PGPPPPPPPPPPGG

SEQ ID NO: 302 - protein, 14 aa, synthetic construct
  REGION 1..14: note = linker
GGPPPPPPPPPPGG

SEQ ID NO: 303 - protein, 14 aa, synthetic construct
  REGION 1..14: note = linker
PPGPPPPPPPPPGG

SEQ ID NO: 304 - protein, 14 aa, synthetic construct
  REGION 1..14: note = linker
GPGPPPPPPPPPGG

SEQ ID NO: 305 - protein, 14 aa, synthetic construct
  REGION 1..14: note = linker
PGGPPPPPPPPPGG

SEQ ID NO: 306 - protein, 14 aa, synthetic construct
  REGION 1..14: note = linker
PPPGPPPPPPPPGG

SEQ ID NO: 307 - protein, 14 aa, synthetic construct
  REGION 1..14: note = linker
GPPGPPPPPPPPGG

SEQ ID NO: 308 - protein, 14 aa, synthetic construct
  REGION 1..14: note = linker
PGPGPPPPPPPPGG

SEQ ID NO: 309 - protein, 14 aa, synthetic construct
  REGION 1..14: note = linker
PPGGPPPPPPPPGG

SEQ ID NO: 310 - protein, 14 aa, synthetic construct
  REGION 1..14: note = linker
PPPPGPPPPPPPGG

SEQ ID NO: 311 - protein, 14 aa, synthetic construct
  REGION 1..14: note = linker
GPPPGPPPPPPPGG

SEQ ID NO: 312 - protein, 14 aa, synthetic construct
  REGION 1..14: note = linker
PGPPGPPPPPPPGG

SEQ ID NO: 313 - protein, 14 aa, synthetic construct
  REGION 1..14: note = linker
PPGPGPPPPPPPGG

SEQ ID NO: 314 - protein, 14 aa, synthetic construct
  REGION 1..14: note = linker
PPPGGPPPPPPPGG

SEQ ID NO: 315 - protein, 14 aa, synthetic construct
  REGION 1..14: note = linker
PPPPPGPPPPPPGG

SEQ ID NO: 316 - protein, 14 aa, synthetic construct
  REGION 1..14: note = linker
GPPPPGPPPPPPGG

SEQ ID NO: 317 - protein, 14 aa, synthetic construct
  REGION 1..14: note = linker
PGPPPGPPPPPPGG

SEQ ID NO: 318 - protein, 14 aa, synthetic construct
  REGION 1..14: note = linker
PPGPPGPPPPPPGG

SEQ ID NO: 319 - protein, 14 aa, synthetic construct
  REGION 1..14: note = linker
PPPGPGPPPPPPGG

SEQ ID NO: 320 - protein, 14 aa, synthetic construct
  REGION 1..14: note = linker
PPPPGGPPPPPPGG

SEQ ID NO: 321 - protein, 14 aa, synthetic construct
  REGION 1..14: note = linker
PPPPPPGPPPPPGG

SEQ ID NO: 322 - protein, 14 aa, synthetic construct
  REGION 1..14: note = linker
GPPPPPGPPPPPGG

SEQ ID NO: 323 - protein, 14 aa, synthetic construct
  REGION 1..14: note = linker
PGPPPPGPPPPPGG

SEQ ID NO: 324 - protein, 14 aa, synthetic construct
  REGION 1..14: note = linker
PPGPPPGPPPPPGG

SEQ ID NO: 325 - protein, 14 aa, synthetic construct
  REGION 1..14: note = linker
PPPGPPGPPPPPGG

SEQ ID NO: 326 - protein, 14 aa, synthetic construct
  REGION 1..14: note = linker
PPPPGPGPPPPPGG

SEQ ID NO: 327 - protein, 14 aa, synthetic construct
  REGION 1..14: note = linker
PPPPPGGPPPPPGG

SEQ ID NO: 328 - protein, 14 aa, synthetic construct
  REGION 1..14: note = linker
PPPPPPPGPPPPGG

SEQ ID NO: 329 - protein, 14 aa, synthetic construct
  REGION 1..14: note = linker
GPPPPPPGPPPPGG

SEQ ID NO: 330 - protein, 14 aa, synthetic construct
  REGION 1..14: note = linker
PGPPPPPGPPPPGG

SEQ ID NO: 331 - protein, 14 aa, synthetic construct
  REGION 1..14: note = linker
PPGPPPPGPPPPGG

SEQ ID NO: 332 - protein, 14 aa, synthetic construct
  REGION 1..14: note = linker
PPPGPPPGPPPPGG

SEQ ID NO: 333 - protein, 14 aa, synthetic construct
  REGION 1..14: note = linker
PPPPGPPGPPPPGG

SEQ ID NO: 334 - protein, 14 aa, synthetic construct
  REGION 1..14: note = linker
PPPPPGPGPPPPGG

SEQ ID NO: 335 - protein, 14 aa, synthetic construct
  REGION 1..14: note = linker
PPPPPPGGPPPPGG

SEQ ID NO: 336 - protein, 14 aa, synthetic construct
  REGION 1..14: note = linker
PPPPPPPPGPPPGG

SEQ ID NO: 337 - protein, 14 aa, synthetic construct
  REGION 1..14: note = linker
GPPPPPPPGPPPGG

SEQ ID NO: 338 - protein, 14 aa, synthetic construct
  REGION 1..14: note = linker
PGPPPPPPGPPPGG

SEQ ID NO: 339 - protein, 14 aa, synthetic construct
  REGION 1..14: note = linker
PPGPPPPPGPPPGG

SEQ ID NO: 340 - protein, 14 aa, synthetic construct
  REGION 1..14: note = linker
PPPGPPPPGPPPGG

SEQ ID NO: 341 - protein, 14 aa, synthetic construct
  REGION 1..14: note = linker
PPPPGPPPGPPPGG

SEQ ID NO: 342 - protein, 14 aa, synthetic construct
  REGION 1..14: note = linker
PPPPPGPPGPPPGG

SEQ ID NO: 343 - protein, 14 aa, synthetic construct
  REGION 1..14: note = linker
PPPPPPGPGPPPGG

SEQ ID NO: 344 - protein, 14 aa, synthetic construct
  REGION 1..14: note = linker
PPPPPPPGGPPPGG

SEQ ID NO: 345 - protein, 14 aa, synthetic construct
  REGION 1..14: note = linker
PPPPPPPPPGPPGG

SEQ ID NO: 346 - protein, 14 aa, synthetic construct
  REGION 1..14: note = linker
GPPPPPPPPGPPGG

SEQ ID NO: 347 - protein, 14 aa, synthetic construct
  REGION 1..14: note = linker
PGPPPPPPPGPPGG

SEQ ID NO: 348 - protein, 14 aa, synthetic construct
  REGION 1..14: note = linker
PPGPPPPPPGPPGG

SEQ ID NO: 349 - protein, 14 aa, synthetic construct
  REGION 1..14: note = linker
PPPGPPPPPGPPGG

SEQ ID NO: 350 - protein, 14 aa, synthetic construct
  REGION 1..14: note = linker
PPPPGPPPPGPPGG

SEQ ID NO: 351 - protein, 14 aa, synthetic construct
  REGION 1..14: note = linker
PPPPPGPPPGPPGG

SEQ ID NO: 352 - protein, 14 aa, synthetic construct
  REGION 1..14: note = linker
PPPPPPGPPGPPGG

SEQ ID NO: 353 - protein, 14 aa, synthetic construct
  REGION 1..14: note = linker
PPPPPPPGPGPPGG

SEQ ID NO: 354 - protein, 14 aa, synthetic construct
  REGION 1..14: note = linker
PPPPPPPPGGPPGG

SEQ ID NO: 355 - protein, 14 aa, synthetic construct
  REGION 1..14: note = linker
PPPPPPPPPPGPGG

SEQ ID NO: 356 - protein, 14 aa, synthetic construct
  REGION 1..14: note = linker
GPPPPPPPPPGPGG

SEQ ID NO: 357 - protein, 14 aa, synthetic construct
  REGION 1..14: note = linker
PGPPPPPPPPGPGG

SEQ ID NO: 358 - protein, 14 aa, synthetic construct
  REGION 1..14: note = linker
PPGPPPPPPPGPGG

SEQ ID NO: 359 - protein, 14 aa, synthetic construct
  REGION 1..14: note = linker
PPPGPPPPPPGPGG

SEQ ID NO: 360 - protein, 14 aa, synthetic construct
  REGION 1..14: note = linker
PPPPGPPPPPGPGG

SEQ ID NO: 361 - protein, 14 aa, synthetic construct
  REGION 1..14: note = linker
PPPPPGPPPPGPGG

SEQ ID NO: 362 - protein, 14 aa, synthetic construct
  REGION 1..14: note = linker
PPPPPPGPPPGPGG

SEQ ID NO: 363 - protein, 14 aa, synthetic construct
  REGION 1..14: note = linker
PPPPPPPGPPGPGG

SEQ ID NO: 364 - protein, 14 aa, synthetic construct
  REGION 1..14: note = linker
PPPPPPPPGPGPGG

SEQ ID NO: 365 - protein, 14 aa, synthetic construct
  REGION 1..14: note = linker
PPPPPPPPPGGPGG

SEQ ID NO: 366 - protein, 14 aa, synthetic construct
  REGION 1..14: note = linker
PPPPPPPPPPPGGG

SEQ ID NO: 367 - protein, 14 aa, synthetic construct
  REGION 1..14: note = linker
GPPPPPPPPPPGGG

SEQ ID NO: 368 - protein, 14 aa, synthetic construct
  REGION 1..14: note = linker
PGPPPPPPPPPGGG

SEQ ID NO: 369 - protein, 14 aa, synthetic construct
  REGION 1..14: note = linker
PPGPPPPPPPPGGG

SEQ ID NO: 370 - protein, 14 aa, synthetic construct
  REGION 1..14: note = linker
PPPGPPPPPPPGGG

SEQ ID NO: 371 - protein, 14 aa, synthetic construct
  REGION 1..14: note = linker
PPPPGPPPPPPGGG

SEQ ID NO: 372 - protein, 14 aa, synthetic construct
  REGION 1..14: note = linker
PPPPPGPPPPPGGG

SEQ ID NO: 373 - protein, 14 aa, synthetic construct
  REGION 1..14: note = linker
PPPPPPGPPPPGGG

SEQ ID NO: 374 - protein, 14 aa, synthetic construct
  REGION 1..14: note = linker
PPPPPPPGPPPGGG

SEQ ID NO: 375 - protein, 14 aa, synthetic construct
  REGION 1..14: note = linker
PPPPPPPPGPPGGG

SEQ ID NO: 376 - protein, 14 aa, synthetic construct
  REGION 1..14: note = linker
PPPPPPPPPGPGGG

SEQ ID NO: 377 - protein, 14 aa, synthetic construct
  REGION 1..14: note = linker
PPPPPPPPPPGGGG

SEQ ID NO: 378 - protein, 8 aa, synthetic construct
  REGION 1..8: note = linker
PPPPPPPG

SEQ ID NO: 379 - protein, 9 aa, synthetic construct
  REGION 1..9: note = linker
PPPPPPPPG

SEQ ID NO: 380 - protein, 10 aa, synthetic construct
  REGION 1..10: note = linker
PPPPPPPPPG

SEQ ID NO: 381 - protein, 11 aa, synthetic construct
  REGION 1..11: note = linker
PPPPPPPPPPG

SEQ ID NO: 382 - protein, 12 aa, synthetic construct
  REGION 1..12: note = linker
PPPPPPPPPPPG

SEQ ID NO: 383 - protein, 13 aa, synthetic construct
  REGION 1..13: note = linker
PPPPPPPPPPPPG

SEQ ID NO: 384 - protein, 14 aa, synthetic construct
  REGION 1..14: note = linker
PPPPPPPPPPPPPG

SEQ ID NO: 385 - protein, 15 aa, synthetic construct
  REGION 1..15: note = linker
PPPPPPPPPPPPPPG

SEQ ID NO: 386 - protein, 16 aa, synthetic construct
  REGION 1..16: note = linker
PPPPPPPPPPPPPPPG

SEQ ID NO: 387 - protein, 17 aa, synthetic construct
  REGION 1..17: note = linker
PPPPPPPPPPPPPPPPG

SEQ ID NO: 388 - protein, 18 aa, synthetic construct
  REGION 1..18: note = linker
PPPPPPPPPPPPPPPPPG

SEQ ID NO: 389 - protein, 21 aa, synthetic construct
  REGION 1..21: note = linker
PPPPPPPPPPPPPPPPPPPPG

SEQ ID NO: 390 - protein, 29 aa, synthetic construct
  REGION 1..29: note = Linker
PPSGGNGGTGTPTSTAPGSGQTSPGGGSG

SEQ ID NO: 391 - protein, 278 aa, Thielavia terrestris [sequence omitted]

SEQ ID NO: 392 - protein, 212 aa, synthetic construct
  REGION 1..212: note = CD of SEQ ID NO:391
ASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKRNDDSSFPVFT

SEQ ID NO: 393 - protein, 37 aa, synthetic construct
  REGION 1..37: note = CBM of SEQ ID NO:391
CTSQKWAQCGGIGFSGCTTCVSGTTCQKLNDYYSQCL

SEQ ID NO: 394 - protein, 212 aa, synthetic construct
  REGION 1..212: note = Catalytic domain of EG variants
ASGSGQSTRYWDCCKPSCAWPGKAAVSQPVYSCDANFQRLSDFNVQSGCNGGSAYACADQTPWAVNDNLAYGFAATSIAGGSESSWCCACYALTFTSGPVAGKTMVVQSTSTGGDLGSNHFDIAMPGGGVGIFDGCSSQFGGLPGDRYGGISSRDQCDSFPAPLKPGCYWRFDWFQNADNPTVTFQQVQCPAEIVARSGCKRNDDSSFPVFT

SEQ ID NO: 395 - protein, 37 aa, synthetic construct
  REGION 1..37: note = CBM of EG variants
CTTQKWGQCGGIGYTGCTNCVAGTTCTQLNPWYHQCR

SEQ ID NO: 396 - protein, 37 aa, synthetic construct
  REGION 1..37: note = AB-CBM w 3 mutations
CTTQKWGQCGGIGYTGCTNCVNGTTCTQLNPWYHQCR

SEQ ID NO: 397 - protein, 263 aa, synthetic construct
  REGION 1..263: note = Candidate CZP4073
ASGSGQSTRYWDCCKPSCAWPGKAAVSQPVYSCDANFQRLSDFNVQSGCNGGSAYACADQTPWAVNDNLAYGFAATSIAGGSESSWCCACYALTFTSGPVAGKTMVVQSTSTGGDLGSNHFDIAMPGGGVGIFDGCSSQFGGLPGDRYGGISSRDQCDSFPAPLKPGCYWRFDWFQNADNPTVTFQQVQCPAEIVARSGCKRNDDSSFPVFTPPPPPGPPPPPPPGCTTQKWGQCGGIGYTGCTNCVAGTTCTQLNPWYHQCR

SEQ ID NO: 398 - protein, 263 aa, synthetic construct
  REGION 1..263: note = Candidate 4074
ASGSGQSTRYWDCCKPSCAWPGKAAVSQPVYSCDANFQRLSDFNVQSGCNGGSAYACADQTPWAVNDNLAYGFAATSIAGGSESSWCCACYALTFTSGPVAGKTMVVQSTSTGGDLGSNHFDIAMPGGGVGIFDGCSSQFGGLPGDRYGGISSRDQCDSFPAPLKPGCYWRFDWFQNADNPTVTFQQVQCPAEIVARSGCKRNDDSSFPVFTPPGPPPPPGPPPPGCTTQKWGQCGGIGYTGCTNCVAGTTCTQLNPWYHQCR

SEQ ID NO: 399 - protein, 263 aa, synthetic construct
  REGION 1..263: note = Candidate4122
ASGSGQSTRYWDCCKPSCAWPGKAAVSQPVYSCDANFQRLSDFNVQSGCNGGSAYACADQTPWAVNDNLAYGFAATSIAGGSESSWCCACYALTFTSGPVAGKTMVVQSTSTGGDLGSNHFDIAMPGGGVGIFDGCSSQFGGLPGDRYGGISSRDQCDSFPAPLKPGCYWRFDWFQNADNPTVTFQQVQCPAEIVARSGCKRNDDSSFPVFTPPPPPPPPGPPPPGCTTQKWGQCGGIGYTGCTNCVAGTTCTQLNPWYHQCR

SEQ ID NO: 400 - protein, 266 aa, synthetic construct
  REGION 1..266: note = Endoglucanase from FSW
ASGSGQSTRYWDCCKPSCAWPGKAAVSQPVYSCDANFQRLSDFNVQSGCNGGSAYACADQTPWAVNDNLAYGFAATSIAGGSESSWCCACYALTFTSGPVAGKTMVVQSTSTGGDLGSNHFDIAMPGGGVGIFDGCSSQFGGLPGDRYGGISSRDQCDSFPAPLKPGCYWRFDWFQNADNPTVTFQQVQCPAEIVARSGCKRNDDSSFPVFTPPPGPPPGPPPGPPPPGCTTQKWGQCGGIGYTGCTNCVAGTTCTQLNPWYHQCR

SEQ ID NO: 401 - protein, 263 aa, synthetic construct
  REGION 1..263: note = Endoglucanase from FSW
ASGSGQSTRYWDCCKPSCAWPGKAAVSQPVYSCDANFQRLSDFNVQSGCNGGSAYACADQTPWAVNDNLAYGFAATSIAGGSESSWCCACYALTFTSGPVAGKTMVVQSTSTGGDLGSNHFDIAMPGGGVGIFDGCSSQFGGLPGDRYGGISSRDQCDSFPAPLKPGCYWRFDWFQNADNPTVTFQQVQCPAEIVARSGCKRNDDSSFPVFTPPGPPPPPPPPPPGCTTQKWGQCGGIGYTGCTNCVNGTTCTQLNPWYHQCR

SEQ ID NO: 402 - protein, 311 aa, synthetic construct
  REGION 1..311: note = Protease for linker stability assay
AVPSTQTPWGIKSIYNDQSITKTTGGKGIKVAVLDTGVYTSHLDLAGSAEQCKDFTQSNPLVDGSCTDRQGHGTHVAGTVLAHGGSNGQGVYGVAPQAKLWAYKVLGDKGEGYSDDIAAAIRHVADEASRTGSKVVINMSLGSSAKDSLIASAVDYAYGKGVLIVAAAGNEGPKPNTIGYPAGFVNAVAVAALENVQEKGTYRVADFSSRGNPATAGDYIIQERDIEVSAPGASVESTWYTGGYNTISGTSMATPHVAGLAAKIWSANTSLSHSQLRTELQNRAKVYDIKGGIGAGPGDDYASGFGYPRVK

SEQ ID NO: 403 - protein, 263 aa, synthetic construct
  REGION 1..263: note = Used to explain notation in Examples
ASGSGQSTRYWDCCKPSCAWPGKAAVSQPVYSCDANFQRLSDFNVQSGCNGGSAYACADQTPWAVNDNLAYGFAATSIAGGSESSWCCACYALTFTSGPVAGKTMVVQSTSTGGDLGSNHFDIAMPGGGVGIFDGCSSQFGGLPGDRYGGISSRDQCDSFPAPLKPGCYWRFDWFQNADNPTVTFQQVQCPAEIVARSGCKRNDDSSFPVFTGPPPPPPPPPPPPGCTTQKWGQCGGIGYTGCTNCVAGTTCTQLNPWYHQCR

SEQ ID NO: 404 - protein, 17 aa, synthetic construct
  REGION 1..17: note = Linker w 3 P to G substitutions
PPPGPPPGPPPGPPPPG

SEQ ID NO: 405 - protein, 257 aa, synthetic construct
  REGION 1..257: note = P7G variant
ASGSGQSTRYWDCCKPSCAWPGKAAVSQPVYSCDANFQRLSDFNVQSGCNGGSAYACADQTPWAVNDNLAYGFAATSIAGGSESSWCCACYALTFTSGPVAGKTMVVQSTSTGGDLGSNHFDIAMPGGGVGIFDGCSSQFGGLPGDRYGGISSRDQCDSFPAPLKPGCYWRFDWFQNADNPTVTFQQVQCPAEIVARSGCKRNDDSSFPVFTPPPPPPPGCTTQKWGQCGGIGYTGCTNCVAGTTCTQLNPWYHQCR

SEQ ID NO: 406 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISAPASYANAMAVGATDQNNNRASFSQYGPGLDIVAPGVNVQSTYPGSTYASLNGTSMATPHVAGAAALVKQKNPSWSNVQIRNHLKNTATSLGSTNLYGSGLVNAEAATR

SEQ ID NO: 407 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 408 - protein, 269 aa, Bacillus lentus
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNILSTWPGSTYASLNGTSMATPHVAGAAALVKQKNPSWSNVQIRNHLKNTATSLGDTWEYGSGLVNAEAATR

SEQ ID NO: 409 - protein, 281 aa, Acremonium thermophilum
LDGKSTRYWDCCKPSCGWAGKAPVNQPVFSCSADWQRISDFNAKSGCDGGNAYSCADQTPWAVNEQFSYGFAATAISGGSEQSWCCACYALTFNSGPVAGKTMVVQSTSTGGDLGENHFDLAIPGGGVGIFNGCQSQFGGLPGAQYGGIQDRSQCSSFPAPLQPGCYWRFDWFQNADNPTFTFQRVQCPSELTSRTGCKRDDDANYPVFSPPSGRPSGSSPPGGSPPGTTTTRRPATTTGSSPTPTQSHYGQCGGIGYSGPTVCASGTTCQVLNPYYSQCL

SEQ ID NO: 410 - protein, 15 aa, synthetic construct
  REGION 1..15: note = construct
PPPGPPPPPPPPPPG

SEQ ID NO: 411 - protein, 10 aa, synthetic construct
  REGION 1..10: note = construct
PPGPPGPPPG

SEQ ID NO: 412 - protein, 9 aa, synthetic construct
  REGION 1..9: note = construct
PPPGPPPPG

SEQ ID NO: 413 - protein, 11 aa, synthetic construct
  REGION 1..11: note = construct
PPPGPPPPPPG

SEQ ID NO: 414 - protein, 12 aa, synthetic construct
  REGION 1..12: note = Construct
PPPGPPPPPPPG

SEQ ID NO: 415 - protein, 10 aa, synthetic construct
  REGION 1..10: note = construct
PPPGPPPPPG

SEQ ID NO: 416 - protein, 13 aa, synthetic construct
  REGION 1..13: note = construct
PPGPPPPPPPPPG

SEQ ID NO: 417 - protein, 16 aa, synthetic construct
  REGION 1..16: note = construct
PPPGPPPPPPPPPPPG

SEQ ID NO: 418 - protein, 17 aa, synthetic construct
  REGION 1..17: note = construct
PPPGPPPPPPPPPPPPG

SEQ ID NO: 419 - protein, 18 aa, synthetic construct
  REGION 1..18: note = construct
PPPGPPPPPPPPPPPPPG

SEQ ID NO: 420 - protein, 18 aa, synthetic construct
  REGION 1..18: note = construct
PPGPPPPGPPPPPPPPPG

SEQ ID NO: 421 - protein, 18 aa, synthetic construct
  REGION 1..18: note = construct
PPGGPPPPPPPPPPPPPG

SEQ ID NO: 422 - protein, 14 aa, synthetic construct
  REGION 1..14: note = construct
PPPPPPPPVPPPPG

SEQ ID NO: 423 - protein, 14 aa, synthetic construct
  REGION 1..14: note = construct
PPPPPPPPNPPPPG

SEQ ID NO: 424 - protein, 14 aa, synthetic construct
  REGION 1..14: note = construct
PPPPPPPPFPPPPG

SEQ ID NO: 425 - protein, 14 aa, synthetic construct
  REGION 1..14: note = construct
PPPPPPPPDPPPPG

SEQ ID NO: 426 - protein, 268 aa, Bacillus lentus
QTVPWGISFINTQQAHNRGIFGNGARVAVLDTGIASHPDLRIAGGASFISSEPSYHDNNGHGTHVAGTIAALNNSIGVLGVAPSADLYAVKVLDRNGSGSLASVAQGIEWAINNNMHIINMSLGSTSGSSTLELAVNRANNAGILLVGAAGNTGRQGVNYPARYSGVMAVAAVDQNGQRASFSTYGPEIEISAPGVNVNSTYTGNRYVSLSGTSMATPHVAGVAALVKSRYPSYTNNQIRQRINQTATYLGSPSLYGNGLVHAGRATQ

SEQ ID NO: 427 - protein, 274 aa, Bacillus lentus
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 428 - protein, 274 aa, Bacillus subtilis
AQTVPYGIPLIKADKVQAQGFKGANVKVAVLDTGIQASHPDLRVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASRVRNRLSSTATYLGSSFYYGKGLINVEAAAQ

SEQ ID NO: 429 - protein, 274 aa, Bacillus subtilis
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSSGSGSYSGIVSGIEWATTNGMDVINMSLGSPSGSTAMKQAVDNAYARGVVVVAAAGNSGSSGNTNTIQYPAKYDSVIAVGAVDSNSQRASFSSVGAELEVMAPGVGVYSTYPTNTYATLSGTSMASPHVAGAAALILSKHPNLSASQVRNRLSSTATYLGPSFYYGKGLINVEAAAQ

SEQ ID NO: 430 - protein, 268 aa, Bacillus subtilis
QTVPWGITRVQAPAVHNRGITGSGVRVAILDSGISAHSDLNIRGGASFVPGEPTTADLNGHGTHVAGTVAALDNSIGVIGVAPNAELYAVKVLGANGSGSVSGIAQGLEWAATNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 431 - protein, 269 aa, Bacillus gibsonii
QQTVPWGITRVQAPTVHNRGITGSGVKVAILDTGIAQHSDLTIRGGASFVPGESTTADLNGHGTHVAGTVAALDNSIGVIGVAPSADLYAVKVLGADGRGSVSGIAQGLEWAATNNMHIANMSLGSDAPSTTLERAVNYATSRGVLVIAATGNNGTGSIGYPARYANAMAVGATDQNNRRASFSQYGTGIDIVAPGVGIQSTYLNNSYASLDGTSMATPHVAGVAALVKQKNPSWNATQIRNHLKNTATNLGNSSQFGSGLVNADAATR

SEQ ID NO: 432 - protein, 269 aa, Bacillus gibsonii
QQTVPWGITRVQAPTVHNRGITGSGVKVAILDTGIAQHSDLTIRGGASFVPGESTTADLNGHGTHVAGTVAALNNSIGVIGVAPSADLYAVKVLGANGRGSVSGIAQGLEWAATNNMHIANMSLGSDAPSTTLERAVNYATSRGVLVIAATGNNGTGSIGYPARYANAMAVGATDQNNRRASFSQYGTGIDIVAPGVGIQSTYLNNSYASLDGTSMATPHVAGVAALVKQKNPSWNATQIRNHLKNTATNLGNSSQFGSGLVNADAATR

SEQ ID NO: 433 - protein, 275 aa, Bacillus amyloliquefaciens [sequence omitted]

SEQ ID NO: 434 - protein, 275 aa, Bacillus amyloliquefaciens
AQSVPYGVSQIKAPALHSQGYTGSNVKVAVIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNKYGALNGTSMASPHVAGAAALILSKHPNWTNTQVRSSLENTTTKLGDSFYYGKGLINVQAAAQ

SEQ ID NO: 435 - protein, 275 aa, Bacillus amyloliquefaciens
AQSVPYGVSQIKAPALHSQGYTGGNVKVAVIDSGIDSSHPDLKVAGGASMVPGETNPFQDNNSHGTHVAGTVAALNNNIGVLGVAPSASLYAVKVLGADGNGQYSWIINGIEWAIANNMDVINMSLGSPSGSAALKAAVDKAVASGVVVVAAAGNEGTSGSSSTVGYPGKYPSVIAVGAVDSSNQRASFSSVGPELDVMAPGVSIQSTLPGNKYGAQNGTSMASPHVAGAAALILSKHPNWTNTQVRSSLENTTTKLGDSFYYGKGLINVQAAAQ

SEQ ID NO: 436 - protein, 275 aa, Bacillus amyloliquefaciens
AQSVPYGVSQIKAPALHSQGYTGGNVKVAVIDSGIDSSHPDLKVAGGASMVPGETNPFQDNNSHGTHVAGTVAALNNNIGVLGVAPSASLYAVKVLGADGNGQYSWIINGIEWAIANNMDVINMSLGAPSGSAALKAAVDKAVASGVVVVAAAGNEGTSGSSSTVGYPGKYPSVIAVGAVDSSNQRASFSSVGPELDVMAPGVSIQSTLPGNKYGAQNGTSMASPHVAGAAALILSKHPNWTNTQVRSSLENTTTKLGDSFYYGKGLINVQAAAQ

SEQ ID NO: 437 - protein, 300 aa, Bacillus amyloliquefaciens
AATTGTGTTLKGKTVSLNISSESGKYVLRDLSKPTGTQIITYDLQNREYNLPGTLVSSTTNQFTTSSQRAAVDAHYNLGKVYDYFYQKFNRNSYDNKGGKIVSSVHYGSRYNNAAWIGDQMIYGDGDGSFFSPLSGSMDVTAHEMTHGVTQETANLNYENQPGALNESFSDVFGYFNDTEDWDIGEDITVSQPALRSLSNPTKYGQPDNFKNYKNLPNTDAGDYGGVHTNSGIPNKAAYNTITKIGVNKAEQIYYRALTVYLTPSSTFKDAKAALIQSARDLYGSQDAASVEAAWNAVGL

SEQ ID NO: 438 - protein, 270 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGAADGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

(19) United States
(12) Patent Application Publication
(10) Pub. No.: US 2024/0417709 A1
(43) Pub. Date: Dec. 19, 2024

(54) ENDOGLUCANASE WITH IMPROVED STABILITY

(71) Applicant: Novozymes A/S, Bagsvaerd (DK)

(72) Inventors: Kenneth Jensen, Oelsted (DK); Svend Gunnar Kaasgaard, Skovlunde (DK); Frank Rasmussen, Roskilde (DK)

(73) Assignee: Novozymes A/S, Bagsvaerd (DK)

(21) Appl. No.: 18/700,134

(22) PCT Filed: Oct. 10, 2022
(86) PCT No.: PCT/EP2022/078079

§ 371 Date: Apr. 10, 2024

(30) Foreign Application Priority Data
Oct. 12, 2021 (DK) PA202100976

(51) Int. Cl. C12N 9/42; C11D 3/386

(52) U.S. Cl. CPC C12N 9/2437; C11D 3/38645; C11D 2111/12

(57) ABSTRACT

Disclosed are endoglucanases having improved stability, e.g., in the presence of a prote-ase, and the use of such endoglucanases in detergent applications, such as laundry or dish wash.

### REFERENCE TO SEQUENCE LISTING

This application contains a Sequence Listing in computer readable form. The computer readable form is incorporated herein by reference.

### FIELD OF THE INVENTION

The present invention relates to polypeptides having endoglucanase activity. The polypeptides may have improved stability and/or wash performance, in particular stability and/or wash performance in the presence of detergent and/or protease. Further, the invention relates to compositions, such as softeners and detergent compositions, comprising a stabilized polypeptide having endoglucanase activity as well as the use of said endoglucanases in detergent applications, such as laundry or dish wash

### BACKGROUND OF THE INVENTION

Endoglucanases generally contain a catalytic domain and one or more carbohydrate binding modules (CBM), which are joined by a linker region. Linkers are generally flexible connectors that provide connectivity between structured domains, but their functional role is largely unknown.

Endoglucanases have for many years been used in detergents due to their observed benefits in the laundry process, such as color clarification, prevent redeposition, anti-pilling/pill removal and improved whiteness, and are characterized by their ability to cleave the 1,4-beta-glycosidic linkages in cellulose molecules into smaller molecules.

Most commercial detergent compositions comprise proteases that improve the removal of many common stains. However, proteases also degrade other proteins available in the washing solution, including other enzymes such as endoglucanases. In particular, the linker in endoglucanases are susceptible to proteolytic activity. It is therefore desirable to provide endoglucanases having increased stability in the presence of proteases, in particular endoglucanases where the linker is less prone to proteolytic activity.

WO 2020/208056 discloses variants of glycoside hydrolases, such as endoglucanases, with a proline-rich linker region having improved stability in comparison with the parent endoglucanase in an aqueous composition comprising a protease, but the wash performance of the endoglucanase variants before storage is less compared to the parent endoglucanase.

### SUMMARY OF THE INVENTION

The inventors of the present invention have surprisingly found that introduction of certain proline-rich linkers makes it possible to obtain polypeptides with endoglucanase activity that at the same time have good stability in the presence of proteases while maintaining a good wash performance.

Accordingly, the invention provides endoglucanases that comprises a catalytic domain, a proline-rich linker region, and a carbohydrate binding module (CBM), wherein the linker is of the form (P/X)ₐG wherein the value of a is in the range 8-16, X is selected from the amino acids G, V, N, F and D, and (P/X)ₐ specifies that each position in the linker is selected from the group consisting of the amino acids G, V, N, F, D and P, and wherein further the linker comprises at least one and not more than five amino acids selected from the group consisting of G, V, N, F and D.

The endoglucanase may be further characterized in that the catalytic domain has a least 80% sequence identity to SEQ ID NO:394 the CBM has at least 80% sequence identity to SEQ ID NO: 395 or to SEQ ID NO:396.

Compositions, in particular softeners and detergent compositions, such as liquid detergent compositions, comprising the endoglucanases, and the use of such compositions for laundering textiles are also disclosed.

### DEFINITIONS

In accordance with this detailed description, the following definitions apply. Note that the singular forms “a,” “an,” and “the” include plural references unless the context clearly dictates otherwise. Unless defined otherwise or clearly indicated by context, all technical and scientific terms used herein have the same meaning as commonly understood by one of ordinary skill in the art to which this invention belongs.

### Amino Acids

The polypeptides of the present invention comprise amino acids selected from the 20 natural amino acids. To represent the 20 natural amino acids the following nomenclature is used: alanine—ala—A, arginine—arg—R, asparagine—asn—N, aspartic acid—asp—D, cysteine—cys—C, glutamine—gln—Q, glutamic acid—glu—E, glycine—gly—G, histidine—his—H, isoleucine—ile—I, leucine—leu—L, lysine—lys—K, methionine—met—M, phenylalanine—phe—F, proline—pro—P, serine—ser—S, threonine—thr—T, tryptophan—trp—W, tyrosine—tyr—Y and valine—val—V.

### Anti-Pilling

The term “anti-pilling” denotes removal of pills from the textile surface and/or prevention of formation of pills on the textile surface.

### Cellulolytic Enzyme or Cellulase

The term “cellulolytic enzyme” or “cellulase” means one or more (e.g., several) enzymes that hydrolyze a cellulosic material. Such enzymes include endoglucanase(s) (e.g. EC 3.2.1.4), cello-biohydrolase(s), beta-glucosidase(s), or combinations thereof.

The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24:452-481. Total cellulolytic enzyme activity can be measured using insoluble substrates, including Whatman Nº1 filter paper, microcrystalline cellulose, bacterial cellulose, algal cellulose, cotton, pretreated lignocellulose, etc. The most common total cellulolytic activity assay is the filter paper assay using Whatman Nº1 filter paper as the substrate. The assay was established by the International Union of Pure and Applied Chemistry (IUPAC) (Ghose, 1987, Pure Appl. Chem. 59:257-68).

### Cellulosic Material

The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystal-line matrix of parallel glucan chains. Hemicelluloses usually hydrogen bond to cellulose, as well as to other hemicelluloses, which help stabilize the cell wall matrix.

### Carbohydrate Binding Module (CBM)

The term “carbohydrate binding module” means the region within a carbohydrate-active enzyme that provides carbohydrate-binding affinity (Boraston et al., 2004, Biochem. J. 383:769-781). A majority of known carbohydrate binding modules (CBMs) are contiguous amino acid sequences with a discrete fold. The carbohydrate binding module (CBM) is typically found either at the N-terminal or at the C-terminal extremity of an enzyme. Some CBMs are known to have specificity for cellulose.

Exemplary CBM families useful according to the invention are those of CBM family 1, 4, 17, 28, 30, 44, 72 and 79. Again, with reference to cazy.org/Carbohydrate-Binding-Modules, CBM Family 1 includes modules of approximately 40 residues found almost exclusively in fungi. The cellulose-binding function has been demonstrated in many cases and appears to be mediated by three aromatic residues separated by about 10.4 angstrom and which form a flat surface. CBM family 4 includes modules of approximately 150 residues found in bacterial enzymes. Binding of these modules has been demonstrated with xylan, beta-1,3-glucan, beta-1,3-1,4-glucan, beta-1,6-glucan and amorphous cellulose but not with crystalline cellulose. CBM family 17 includes modules of approximately 200 residues. Binding to amorphous cellulose, cellooligosaccharides and derivatized cellulose has been demonstrated. Regarding CBM family 28, the module from the endo-1,4-glucanase of Bacillus sp. 1139 binds to non-crystalline cellulose, cellooligosaccharides, and beta-(1,3)(1,4)-glucans. For CBM Family 30, binding to cellulose has been demonstrated for the N-terminal module of Fibrobacter succinogenes CelF. The C-terminal CBM44 module of the Clostridium thermocellum enzyme has been demonstrated to bind equally well cellulose and xyloglucan. CBM Family 72 includes modules of 130-180 residues found at the C-terminus glycoside hydrolases from various families, sometimes as tandem repeats. The CBM72 found on an endoglucanase from an uncultivated microorganism was found to bind a broad spectrum of polysaccharides including soluble and insoluble cellulose, beta-1,3/1,4-mixed linked glucans, xylan, and beta-mannan. CBM Family 79 includes modules of approx. 130 residues found so far only in ruminococcal proteins. Binding to various beta-glucans was shown for the R. flavefaciens GH9 enzyme. In the context of the present invention CBM family 1 also referred to as “CBM1” are most preferred. CBM are classified according to Carbohydrate Active enZYmes database available at cazy.org/Carbohydrate-Binding-Modules.html.

### Catalytic Domain

The term “catalytic domain” means the region of an enzyme containing the catalytic site of the enzyme.

### Color Clarification

During washing and wearing loose or broken fibers can accumulate on the surface of the fabrics. One consequence can be that the colors of the fabric appear less bright or less intense because of the surface contaminations. Removal of the loose or broken fibers from the textile will partly restore the original colors and looks of the textile. By the term “color clarification”, as used herein, is meant the partial restoration the visual appearance of the initial colors of textile.

### Detergent Composition

The term “detergent composition” refers to compositions that find use in the removal of undesired compounds from items to be cleaned, such as textiles, dishes, and hard surfaces. The detergent composition may be used to e.g. clean textiles, dishes and hard surfaces for both household cleaning and industrial cleaning and/or for fabric care. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; hard surface cleaning formulations, such as for glass, wood, plastic, ceramic and metal counter tops and windows; carpet cleaners; oven cleaners; fabric fresheners; fabric softeners; and textile and laundry pre-spotters, as well as dish wash detergents). In addition to containing an enzyme of the invention, the detergent formulation may contain one or more additional enzymes (such as amylases, proteases, peroxidases, cellulases, betaglucanases, xyloglucanases, hemicellulases, xanthanases, xanthan lyases, lipases, acyl transferases, phospholipases, esterases, laccases, catalases, aryl esterases, amylases, alpha-amylases, glucoamylases, cutinases, pectinases, pectate lyases, keratinases, reductases, oxidases, phenoloxidases, lipoxygenases, ligninases, carrageenases, pullulanases, tannases, arabinosidases, hyaluronidases, chondroitinases, xyloglucanases, xylanases, pectin acetyl esterases, polygalacturonases, rhamnogalacturonases, other endo-beta-mannanases, exo-beta-mannanases (GH5 and/or GH26), licheninases, phosphodiesterases, pectin methylesterases, cellobiohydrolases, transglutaminases, nucleases, and combinations thereof, or any mixture thereof), and/or detergent components such as surfactants, hydrotropes, builders, co-builders, chelators or chelating agents, bleaching system or bleach components, polymers, fabric hueing agents, fabric conditioners, foam boosters, suds suppressors, dispersants, dye transfer inhibitors, fluorescent whitening agents, perfume, tannish inhibitors, optical brighteners, bactericides, fungicides, soil suspending agents, soil release polymers, anti-redeposition agents, enzyme inhibitors or stabilizers, enzyme activators, antioxidants, and solubilizers. The detergent composition may comprise of one or more of any type of detergent component.

### Endoglucanase

The term “endoglucanase” means an enzyme that catalyzes endohydrolysis of 1,4-beta-D-glycosidic linkages in cellulosic material, such as cellulose, cellulose derivatives (such as carboxymethyl cellulose and hydroxyethyl cellulose), lichenin, beta-1,4 bonds in mixed beta-1,3-beta-1,4 glucans such as cereal beta-D-glucans or xyloglucans, and other plant material containing cellulosic components.

One particularly preferred class of endoglucanase are those of “family GH45”, which are classified as glycoside hydrolase Family 45 according to the terminology of Henrissat et al.,. “Biochem. J. 280:309-316 (1991), as well as the Carbohydrate Active enZYmes database available at cazy.org. GH45 enzymes are endoglucanases of EC 3.2.1.4.

### Engineered

The term “engineered” means a synthetic construct.

### Fabric Care

The term fabric care, also referred to as textile care, refers to treatments that retains or partly or fully restores the properties of the textile, e.g. by color clarification, anti-pilling or prevention of formation of pills on the textile surface.

### Glycoside Hydrolase

The term “glycoside hydrolase” (GH) means an enzyme that catalyzes the hydrolysis of a glycosidic bond between two or more carbohydrates or between a carbohydrate and a non-carbohydrate moiety. For more details, see, for example, Henrissat B., “A classification of glycosyl hydrolases based on amino-acid sequence similarities.” Biochem. J. 280:309-316 (1991), as well as the Carbohydrate Active enZYmes database available at cazy.org.

Exemplary glycoside hydrolase families with reported cellulase activities useful according to the present invention include those of families GH5, GH6, GH7, GH8, GH9, GH12, GH44, GH45, GH48, GH51, GH124, with family GH45 being particularly preferred.

### Hybrid Polypeptide

The term “hybrid polypeptide” means a polypeptide comprising domains from two or more polypeptides from different sources (origins), e.g., a binding module from one polypeptide and a catalytic domain from another polypeptide. The domains may be fused at the N-terminus or the C-terminus. Of particular interest herein are polypeptides comprising a binding module from one polypeptide (which may be naturally occurring or further modified), an engineered linker region, such as a proline-rich linker region, which is a synthetic construct, and a catalytic domain from another polypeptide (which may be naturally occurring or further modified).

### Improved Stability

The term “improved stability” means an enzyme having better stability in the presence of protease relative to the stability of a reference enzyme/parent enzyme, and includes, for example, proteolytic stability, in-detergent storage stability, improved stability during production of the detergent composition, as well as in-wash stability and during rinse. Improved stability may also include stability in enzyme blends, e.g. co-granulates or liquid enzyme formulations with protease.

### Improved Wash Performance

The term “improved wash performance” is defined herein as an enzyme displaying an increased wash performance in a detergent composition relative to the wash performance of a reference enzyme, e.g., by increased color clarification and/or anti-pilling effect, when evaluating the fresh samples and/or after the samples have been stored under the same conditions. The term “improved wash performance” includes wash performance in laundry but also in, e.g., hard surface cleaning such as automated dish wash (ADW).

### Isolated

The term “isolated” means a substance in a form or environment which does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

### Proline-Rich Linker

The term “proline-rich linker” means an amino acid sequence connecting the catalytic core with the CBM. The proline-rich linker may also be referred to simply as “the linker” or “the linker region”. The proline-rich linkers as described herein comprises an amino acid sequence of the form (P/X)ₐG, wherein a is in the range 8-16 and (P/X)ₐ specifies that each position in the linker is selected from the group of amino acids consisting of P, V, D, F, N and G, and the linker comprises 1 to 5 amino acid residue selected from V, D, F, N and G. In an embodiment X is a glycine residue and the linker comprises 2, 3, 4 or 5 glycine residues.

### Purified

The term “purified” means a nucleic acid or polypeptide that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

### Recombinant

The term “recombinant,” when used in reference to a cell, nucleic acid, protein or vector, means that it has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a polypeptide is a recombinant vector. The term “recombinant” is synonymous with “genetically modified” and “transgenic”.

### Sequence Identity

The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the-nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the-nobrief option) is used as the percent identity and is calculated as follows:

(Identical Deoxyribonucleotides×100)/(Length of Alignment−Total Number of Gaps in Alignment)

### Textile

The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used, it is intended to include the broader term textiles as well.

### Variant

The term “variant” means a polypeptide having endoglucanase activity comprising single or multiple amino acid substitutions, deletions, and/or insertions at one or more (e.g., several) positions in parent endoglucanase. E.g., any of SEQ ID NO:397, SEQ ID NO:398, SEQ ID NO: 399, SEQ ID NO:400 and SEQ ID NO:401 can be considered variants of the endoglucanase having SEQ ID NO:391. A “variant” as used herein may also include a hybrid polypeptide.

### Wash Liquor

The term “wash liquor” refers to an aqueous solution containing a detergent composition in dilute form, such as but not limited to a detergent solution containing a laundry detergent composition in dilute form such as the wash liquor in a laundry process.

### Whiteness

The term “Whiteness” is defined herein as a broad term with different meanings in different regions and for different consumers. Loss of whiteness can e.g. be due to greying, yellowing, or removal of optical brighteners/hueing agents. Greying and yellowing can be due to soil redeposition, body soils, coloring from e.g. iron and copper ions or dye transfer. Whiteness might include one or several issues from the list below: colorant or dye effects; incomplete stain removal (e.g. body soils, sebum etc.); redeposition (greying, yellowing or other discolorations of the object) (removed soils re-associate with other parts of textile, soiled or unsoiled); chemical changes in textile during application; and clarification or brightening of colors.

### DETAILED DESCRIPTION OF THE INVENTION

Many proteins are comprised of structured domains connected by linkers. For example, endoglucanases and other glycoside hydrolases (GH) are often found as modular enzymes having one or more catalytic domains, which may be connected to one or more CBMs via a peptide known as a linker, which is sometimes partially glycosylated. The catalytic domain in a endoglucanase is responsible for the hydrolytic degradation of cellulose, while the CBM, when present, works by increasing the effective concentration of enzyme near the substrate surface. In contrast, linkers are generally flexible connectors that provide connectivity between structured domains, but their functional role is largely unknown.

### Endoglucanases of the Present Invention

Endoglucanases are often cleaved in exposed regions or partially or fully degraded by proteases in liquid detergents. Most commonly, the protease cleaves in the linker region of the endoglucanase and thereby reduces the ability of the endoglucanase to remove fuzz and pill and maintaining or restoring the colors of the textile by reducing its ability to bind to the insoluble cellulose substrate. The loss in binding affinity strongly impacts the performance of endoglucanases which is why a protease stable cellulase, in particular cellulase linker stable towards proteolytic activity, is highly valuable in the liquid laundry/dish detergent segment, as well as in softeners.

The invention is directed to cellulases, in particular endoglucanases, having a three-domain structure with the catalytic domain connected to a carbohydrate binding module via a linker. The invention is directed to endoglucanases having peptide stretches that make the linker more stable, i.e., less susceptible to proteolytic cleavage, while maintaining a good wash performance. In an embodiment, the endoglucanase is a GH45 endoglucanase. Polypeptides having N- and/or C-terminal CBMs are contemplated.

One preferred use of the endoglucanases of the invention is in detergents, where proteases typically are included to improve the effect of the detergent. The improved stability of the endoglucanases of the invention means that the endoglucanases can maintain the cellulolytic activity for a longer time either during storage or during the laundry process compared with the parent endoglucanase, and thereby provide an improved wash performance benefit compared with the parent endoglucanase. In the context of the present invention the endoglucanases have an improved stability compared to the commercially available reference (parent) endoglucanase having SEQ ID NO:391 in the presence of a protease and/or a detergent. The improvement in stability can be quantified by determining stability according to the assay described in Example 2 (Linker Stability Assay), whereas the wash performance can be tested as disclosed in Example 7 (Full scale washing) herein.

The dual problem of improving stability while maintaining wash perform of the endoglucanase has been solved by the introduction of a proline rich linker of the form (P/X)ₐG joining the catalytic core and the CBM of the endoglucanase.

In the proline rich linker (P/X)ₐG, P designates a proline residue, G designates a glycine residue, and X designates an amino acid, preferably select from the group consisting of G, V, N, F and D, and (P/X)ₐ specifies that each position in the linker is selected from the group consisting of the amino acids G, V, N, F, D and P. The value of a is in the range 8-16, such as 10-16 or 12-16, such as 10, 11, 12, 13, 14, 15 or 16. (P/X)ₐ must comprise at least one and not more than five amino acids selected from the group consisting of G, V, N, F and D.

In a further preferred embodiment, each of the 1, 2, 3, 4 or 5 amino acids selected from G, V, N, F, D in the proline-rich linker are separated by at least one proline residue, such as 1, 2, 3, 4, 5, 6, 7, 8, 9, 10, 11, 12, 13, 14, 15 proline residues. In a particular embodiment the proline-rich linker comprises the amino acid motif proline-glycine-proline (PGP) one or more times, such as one or two times, preferably two times.

In a particular aspect of the present invention the endoglucanases comprise a proline-rich amino acid sequence of the form (P/G)ₐG, i.e. a linker wherein X is selected as G and (P/G)ₐ designates that each position in the amino acid sequence is selected from proline (P) and glycine (G) residues connecting the catalytic core with the CBM. The value of a is in the range 8-16, such as 10-16 or 12-16, in particular such as 10, 11, 12, 13, 14, 15 or 16. Preferably a has the value 13. For all values of a the (P/G)ₐG linker comprises 2, 3, 4 or 5 glycine residues, preferably 2, 3 or 4 glycine residues, such as 2 or 3 glycine residues, preferably 3 glycine residues. An example of a preferred linker of the form (P/G)ₐG with a=13 is PPPPPGPPPPPPPG (SEQ ID NO:26). Other preferred linkers are PPGPPPPPGPPPPG (SEQ ID NO:97), PPPPPPPPGPPPPG (SEQ ID NO:93), PPPGPPPGPPPGPPPPG (SEQ ID NO:404).

Preferred linkers also include PPPPPPPPVPPPPG (SEQ ID NO: 422), PPPPPPPPNPPPPG (SEQ ID NO: 423), PPPPPPPPFPPPPG (SEQ ID NO: 424) and PPPPPPPPDPPPPG (SEQ ID NO: 425).

For all embodiments of (P/X)ₐG and (P/G)ₐG, the proline-rich linker has preferably at least one proline residue before the first G, V, N, F, D residue.

The catalytic core has at least 80% sequence identity to SEQ ID NO:394, such as at least 85%, 90%, 91%, 92%, 93%, 94%, 95%, 96%, 97%, 98%, 99% or even 100% sequence identity to SEQ ID NO: 394:

ASGSGQSTRY WDCCKPSCAW PGKAAVSQPV YSCDANFQRL
SDFNVQSGCN GGSAYACADQ TPWAVNDNLA YGFAATSIAG
GSESSWCCAC YALTFTSGPV AGKTMVVQST STGGDLGSNH
FDIAMPGGGV GIFDGCSSQF GGLPGDRYGG ISSRDQCDSF
PAPLKPGCYW RFDWFQNADN PTVTFQQVQC PAEIVARSGC
KRNDDSSFPV FT

The CBM has at least 80% sequence identity to SEQ ID NO:395 or to SEQ ID NO:396, such as at least 85%, 90%, 91%, 92%, 93%, 94%, 95%, 96%, 97%, 98%, 99% or even 100% sequence identity to SEQ ID NO:395: CTTQKWGQCG GIGYTGCTNC VAGTTCTQLN PWYHQCR or to SEQ ID NO:396: CTTQKWGQCG GIGYTGCTNC VNGTTCTQLN PWYHQCR

In a particular embodiment the endoglucanase of the present invention has at least 80% sequence identity, such as 85%, 90%, 91%, 92%, 93%, 94%, 95%, 96%, 97%, 98%, 99% or even 100% sequence identity to any of SEQ ID NO:397:

ASGSGQSTRY WDCCKPSCAW PGKAAVSQPV YSCDANFQRL
SDFNVQSGCN GGSAYACADQ TPWAVNDNLA YGFAATSIAG
GSESSWCCAC YALTFTSGPV AGKTMVVQST STGGDLGSNH
FDIAMPGGGV GIFDGCSSQF GGLPGDRYGG ISSRDQCDSF
PAPLKPGCYW RFDWFQNADN PTVTFQQVQC PAEIVARSGC
KRNDDSSFPV FTPPPPPGPP PPPPPGCTTQ KWGQCGGIGY
TGCTNCVAGT TCTQLNPWYH QCR

In a particular embodiment the endoglucanase of the present invention has at least 80% sequence identity, such as 85%, 90%, 91%, 92%, 93%, 94%, 95%, 96%, 97%, 98%, 99% or even 100% sequence identity to any of SEQ ID NO: 398:

ASGSGQSTRY WDCCKPSCAW PGKAAVSQPV YSCDANFQRL
SDFNVQSGCN GGSAYACADQ TPWAVNDNLA YGFAATSIAG
GSESSWCCAC YALTFTSGPV AGKTMVVQST STGGDLGSNH
FDIAMPGGGV GIFDGCSSQF GGLPGDRYGG ISSRDQCDSF
PAPLKPGCYW RFDWFQNADN PTVTFQQVQC PAEIVARSGC
KRNDDSSFPV FTPPGPPPPP GPPPPGCTTQ KWGQCGGIGY
TGCTNCVAGT TCTQLNPWYH QCR

In a particular embodiment the endoglucanase of the present invention has at least 80% sequence identity, such as 85%, 90%, 91%, 92%, 93%, 94%, 95%, 96%, 97%, 98%, 99% or even 100% sequence identity to any of SEQ ID NO:399:

ASGSGQSTRY WDCCKPSCAW PGKAAVSQPV YSCDANFQRL
SDFNVQSGCN GGSAYACADQ TPWAVNDNLA YGFAATSIAG
GSESSWCCAC YALTFTSGPV AGKTMVVQST STGGDLGSNH
FDIAMPGGGV GIFDGCSSQF GGLPGDRYGG ISSRDQCDSF
PAPLKPGCYW RFDWFQNADN PTVTFQQVQC PAEIVARSGC
KRNDDSSFPV FTPPPPPPPP GPPPPGCTTQ KWGQCGGIGY
TGCTNCVAGT TCTQLNPWYH QCR

In a particular embodiment the endoglucanase of the present invention has at least 80% sequence identity, such as 85%, 90%, 91%, 92%, 93%, 94%, 95%, 96%, 97%, 98%, 99% or even 100% sequence identity to any of SEQ ID NO:400:

ASGSGQSTRY WDCCKPSCAW PGKAAVSQPV YSCDANFQRL
SDFNVQSGCN GGSAYACADQ TPWAVNDNLA YGFAATSIAG
GSESSWCCAC YALTFTSGPV AGKTMVVQST STGGDLGSNH
FDIAMPGGGV GIFDGCSSQF GGLPGDRYGG ISSRDQCDSF
PAPLKPGCYW RFDWFQNADN PTVTFQQVQC PAEIVARSGC
KRNDDSSFPV FTPPPGPPPG PPPGPPPPGC TTQKWGQCGG
IGYTGCTNCV AGTTCTQLNP WYHQCR

In a particular embodiment the endoglucanase of the present invention has at least 80% sequence identity, such as 85%, 90%, 91%, 92%, 93%, 94%, 95%, 96%, 97%, 98%, 99% or even 100% sequence identity to any of SEQ ID NO:401:

ASGSGQSTRY WDCCKPSCAW PGKAAVSQPV YSCDANFQRL
SDFNVQSGCN GGSAYACADQ TPWAVNDNLA YGFAATSIAG
GSESSWCCAC YALTFTSGPV AGKTMVVQST STGGDLGSNH
FDIAMPGGGV GIFDGCSSQF GGLPGDRYGG ISSRDQCDSF
PAPLKPGCYW RFDWFQNADN PTVTFQQVQC PAEIVARSGC
KRNDDSSFPV FTPPGPPPPP PPPPPGCTTQ KWGQCGGIGY
TGCTNCVNGT TCTQLNPWYH QCR

In one embodiment of the present invention, the polypeptide of the present invention may be added to a detergent composition in an amount corresponding to 0.001-200 mg of protein, such as 0.005-100 mg of protein, preferably 0.01-50 mg of protein, more preferably 0.05-20 mg of protein, even more preferably 0.1-10 mg of protein per liter of wash liquor.

### Catalytic Domain

The catalytic domain of the endoglucanases of the invention has preferably 80% sequence identity, such as 85%, 90%, 91%, 92%, 93%, 94%, 95%, 96%, 97%, 98%, 99% or even 100% sequence identity to SEQ ID NO:394:

(SEQ ID NO: 394)
ASGSGQSTRYWDCCKPSCAWPGKAAVSQPVYSCDANFQRLSDFNVQSGCN
GGSAYACADQTPWAVNDNLAYGFAATSIAGGSESSWCCACYALTFTSGPV
AGKTMVVQSTSTGGDLGSNHFDIAMPGGGVGIFDGCSSQFGGLPGDRYGG
ISSRDQCDSFPAPLKPGCYWRFDWFQNADNPTVTFQQVQCPAEIVARSGC
KRNDDSSFPVFT.

The catalytic domain may be N-terminal or C-terminal, preferably it is N-terminal.

### Carbohydrate Binding Domain (CBM)

The carbohydrate binding domain of the endoglucanases of the invention has preferably 80% sequence identity, such as 85%, 90%, 91%, 92%, 93%, 94%, 95%, 96%, 97%, 98%, 99% or even 100% sequence identity to SEQ ID NO:395:

(SEQ ID NO: 395)
CTTQKWGQCGGIGYTGCTNCVAGTTCTQLNPWYHQCR,

or the carbohydrate binding domain of the endoglucanases of the invention has preferably 80% sequence identity, such as 85%, 90%, 91%, 92%, 93%, 94%, 95%, 96%, 97%, 98%, 99% or even 100% sequence identity to SEQ ID NO:396:

(SEQ ID NO: 396)
CTTQKWGQCGGIGYTGCTNCVNGTTCTQLNPWYHQCR

The carbohydrate binding domain may be N-terminal or C-terminal, preferably it is C-terminal.

### Stability Assay for Endoglucanases

Stability in presence of protease may be determined by incubating a given endoglucanase under defined conditions in the presence of a protease, measuring the cellulolytic activity after the incubation and comparing it with a sample of the endoglucanase that has not been incubated with protease.

However, in the traditional enzyme stability assays used for testing thermostability, activity measures of the stressed (i.e. stored with the presence of a protease) and unstressed (e.g. freshly prepared or stored under conditions without the presence of a protease) sample typically focused on measuring changes affecting the catalytic site of the enzyme molecule, e.g. by using a small synthetic substrate such as 4-Methylumbelliferyl-beta-cellopentaoside or soluble carboxymethyl cellulose (CMC).

Importantly, however, changes in other properties of the enzyme of interest due to the stress, properties important for the function of the enzyme in the application but not directly affecting the active site of the enzyme, is not necessarily detected in these assays. One such example is the glycosyl hydrolases having a separate catalytic domain and a CBM joined by a linker as e.g. in endoglucanases used for removing fuzz and pills in laundry detergents and textile care products. If the stress affects only the linker and/or CBM part of the molecule but not the catalytic domain part, these changes will not be detected by the traditional assays as described above. The activity, when using a simple substrate such as soluble CMC or 4-methylumbelliferyl-beta-cellopentaoside, will appear to be maintained during the stress but the performance is significantly affected, as the CBM part of the enzyme molecule plays an important role in directing the enzyme to the proper location on the textile to be treated.

As an alternative to traditional enzyme stability assays used for testing thermostability, the importance of the CBM and/or linker for the performance can be tested by comparing the performance of the catalytic domain with that of the catalytic domain with intact linker and CBM. To detect changes in the linker and/or CBM after storage under stressed conditions special measures must be taken when testing if the stress has affected the performance of the enzyme.

This can be done by comparing the performance of the enzymes before and after stress. Alternatively, it can be tested by ensuring that binding of the enzyme to its natural, insoluble substrate, such as cotton linters, is included as part of the assay used for testing the stability, and/or first probing the binding of the enzyme to microcrystalline cellulose or cotton linters and then measure the activity of the enzymes having lost their binding ability to the cellulose compared to the total activity.

Thus, linker stability is measured by incubating the endoglucanase in detergent containing protease, followed by determining the ability of the incubated endoglucanase to bind to cellulose fibers. If the linker or the cellulose binding domain is affected by the protease the binding affinity of the endoglucanase to cellulose fibers will be reduced.

This linker specific assay is illustrated by the conditions described in Example 2.

### Preparation of Variants

The present invention also relates to methods for obtaining a variant having glycoside hydrolase activity, comprising: (a) introducing into a parent glycoside hydrolase one or more substitutions of the mature polypeptide of the parent polypeptide; and (b) recovering the variant.

The variants can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more (e.g., several) mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76:4949-4955; and Barton et al., 1990, Nucleic Acids Res. 18:7349-4966.

Site-directed mutagenesis can also be accomplished in vivo by methods known in the art. See, e.g., U.S. Patent Application Publication No. 2004/0171154; Storici et al., 2001, Nature Biotechnol. 19:773-776; Kren et al., 1998, Nat. Med. 4:285-290; and Calissano and Macino, 1996, Fungal Genet. Newslett. 43:15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432:1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86:2152-2156; WO 95/17413; or WO 95/22625. Other methods that can be used include error-prone PCR, phage display (e.g., Lowman et al., 1991, Biochemistry 30:10832-10837; U.S. Pat. No. 5,223,409; WO 92/06204) and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17:893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling.

Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Host Cells

Production of an endoglucanase of the present invention can take place in recombinant host cells comprising a polynucleotide encoding an endoglucanase of the present invention operably linked to one or more control sequences that eventually direct the production of the endoglucanase. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the variant and its source.

### Methods of Production

The present invention also relates to methods of producing a variant, comprising: (a) cultivating a host cell of the present invention under conditions suitable for expression of the variant; and (b) recovering the variant.

The host cells are cultivated in a nutrient medium suitable for production of the variant using methods known in the art. For example, the cell may be cultivated by shake flask cultivation, or small-scale or large-scale fermentation (including continuous, batch, fed-batch, or solid-state fermentations) in laboratory or industrial fermentors performed in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variant may be detected using methods known in the art that are specific for the variants. These detection methods include, but are not limited to, use of specific antibodies, formation of an enzyme product, or disappearance of an enzyme substrate. For example, an enzyme assay may be used to determine the activity of the variant.

The variant may be recovered using methods known in the art. For example, the variant may be recovered from the nutrient medium by conventional procedures including, but not limited to, collection, centrifugation, filtration, extraction, spray-drying, evaporation, or precipitation.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure variants.

In an alternative aspect, the variant is not recovered, but rather a host cell of the present invention expressing the variant is used as a source of the variant.

### Detergent Compositions

In one embodiment, the invention is directed to detergent compositions comprising an enzyme of the present invention in combination with one or more additional cleaning composition components. The choice of additional components is within the skill of the artisan and includes conventional ingredients, including the exemplary non-limiting components set forth below. Additional, optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

The choice of components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

In one embodiment, the invention is directed to a liquid laundry detergent composition comprising an enzyme of the present invention in combination with one or more additional laundry detergent composition components, specifically a protease. In another embodiment, the invention comprises an ancillary product used in laundry, such as a prespotter or stain removal booster. The present invention also relates to an ADW (Automatic Dish Wash) compositions comprising an enzyme of the present invention in combination with one or more additional ADW composition components. The choice of additional components is within the skill of the artisan and includes conventional ingredients, including the exemplary non-limiting components set forth below.

### Surfactants

Typically, the detergent composition comprises (by weight of the composition) one or more surfactants in the range of 0% to 50%, preferably from 2% to 40%, more preferably from 5% to 35%, more preferably from 7% to 30%, most preferably from 10% to 25%, even most preferably from 15% to 20%. In a preferred embodiment the detergent is a liquid or powder detergent comprising less than 40%, preferably less than 30%, more preferably less than 25%, even more preferably less than 20% by weight of surfactant. The composition may comprise from 1% to 15%, preferably from 2% to 12%, 3% to 10%, most preferably from 4% to 8%, even most preferably from 4% to 6% of one or more surfactants. Preferred surfactants are anionic surfactants, non-ionic surfactants, cationic surfactants, zwitterionic surfactants, amphoteric surfactants, and mixtures thereof. Suitable anionic surfactants are well known in the art and may comprise fatty acid carboxylates (soap), branched-chain, linear-chain and random chain alkyl sulfates or fatty alcohol sulfates or primary alcohol sulfates or alkyl benzenesulfonates such as LAS and LAB or phenylalknesulfonates or alkenyl sulfonates or alkenyl benzenesulfonates or alkyl ethoxysulfates or fatty alcohol ether sulfates or alpha-olefin sulfonate or dodecenyl/tetradecnylsuccinic acid. The anionic surfactants may be alkoxylated. The detergent composition may also comprise from 1 wt % to 10 wt % of non-ionic surfactant, preferably from 2 wt % to 8 wt %, more preferably from 3 wt % to 7 wt %, even more preferably less than 5 wt % of non-ionic surfactant. Suitable non-ionic surfactants are well known in the art and may comprise alcohol ethoxylates, and/or alkyl ethoxylates, and/or alkylphenol ethoxylates, and/or glucamides such as fatty acid N-glucosyl N-methyl amides, and/or alkyl polyglucosides and/or mono- or diethanolamides or fatty acid amides. The detergent composition may also comprise from 0 wt % to 10 wt % of cationic surfactant, preferably from 0.1 wt % to 8 wt %, more preferably from 0.5 wt % to 7 wt %, even more preferably less than 5 wt % of cationic surfactant. Suitable cationic surfactants are well known in the art and may comprise alkyl quaternary ammonium compounds, and/or alkyl pyridinium compounds and/or alkyl quaternary phosphonium compounds and/or alkyl ternary sulphonium compounds. The composition preferably comprises surfactant in an amount to provide from 100 ppm to 5,000 ppm surfactant in the wash liquor during the laundering process. The composition upon contact with water typically forms a wash liquor comprising from 0.5 g/l to 10 g/l detergent composition. Many suitable surface active compounds are available and fully described in the literature, for example, in “Surface-Active Agents and Detergents”, Volumes I and 11, by Schwartz, Perry and Berch. Also preferred are biobased surfactants, which may be wholly biobased (>95% biobased carbon of total carbon according to European standard EN 17035). As used herein biobased surfactants are a commercial or industrial product (other than food or feed) that is composed, in whole or in significant part, of biological products or renewable agricultural materials or forestry materials and/or as established by European standard EN 16575:2014. In particular rhamnolipids and sophorolipids may be used a detergent ingredient.

### Solvent System

For dissolution of the surfactant and other detergent ingredients, a solvent system is needed. Solvents are typically water, alcohols, polyols, sugars and/or mixtures thereof. Preferred solvents are water, glycerol, sorbitol, propylene glycol (MPG, 1,2-propanediol or 1,3-propane diol), dipropylene glycol (DPG), polyethylene glycol family (PEG300-600), hexylene glycol, inositol, mannitol, Ethanol, isopropanol, n-butoxy propoxy propanol, ethanolamines (monoethanol amine, diethanol amines and triethanol amines), sucrose, dextrose, glucose, ribose, xylose, and related mono and di pyranosides and furanosides. The solvent system is present in typically totally 5-90%, 5-60%, 5-40%, 10-30% by weight.

### Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants), however the molecular structure of hydrotropes generally do not favor spontaneous self-aggregation, see e.g. review by Hodgdon and Kaler (2007), Current Opinion in Colloid & Interface Science 12:121-128. The detergent may contain 0-10% by weight, for example 0-5% by weight, such as about 0.5 to about 5%, or about 3% to about 5%, of a hydrotrope.

### Builders and Co-Builders

The detergent composition may contain about 0-65%, 0-20%; or 0.5-5% of a detergent builder or co-builder, or a mixture thereof. In a dish wash detergent, the level of builder is typically 10--65%, particularly 20-40%. The builder and/or co-builder may particularly be a chelating agent that forms water-soluble complexes with Ca and Mg. Any builder and/or co-builder known in the art for use in laundry detergents may be utilized.

### Bleaching Systems

The detergent may contain 0-30% by weight, such as about 1% to about 20%, of a bleaching system. Any bleaching system known in the art for use in laundry detergents may be utilized.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light.

### Dispersants

The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Dye Transfer Inhibiting Agents

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

### Soil Release Polymers

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Anti-Redeposition Agents

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, co-polymers of acrylic acid and maleic acid, and ethoxylated polyethyleneimines. The cellulose based polymers described under soil release polymers above may also function as anti-redeposition agents.

### Rheology Modifiers

Rheology modifiers are structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other Suitable Adjunct Materials Other adjunct materials include, but are not limited to, anti-shrink agents, anti-wrinkling agents, bactericides, binders, carriers, dyes, enzyme stabilizers, fabric softeners, fillers, foam regulators, perfumes, pigments and sod suppressors.

### Additional Enzymes

The detergent additive as well as the detergent composition may comprise one or more [additional] enzymes such as hydrolases (EC 3.-.-.-) such as hydrolases acting on ester bonds (EC 3.1.-.-), glycosidases (EC 3.2.-.-), and hydrolases acting on peptide bonds (EC 3.4.-.-), oxidoreductases (EC 1.-.-.-) such as laccases (EC 1.10.-.-) or peroxidases (EC 1.11.-.-) or lyases (EC 4.-.-.-) such as carbon-oxygen lyases (EC 4.2.-.-). In a specific embodiment the detergent composition may comprise one or more [additional] enzymes such as a protease, lipase, cutinase, an amylase, carbohydrase, cellulase, pectinase, mannanase, arabinase, galactanase, xylanase, oxidase, e.g., a laccase, and/or peroxidase.

In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

### Cellulases

Suitable additional cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259.

### Mannanases

Mannanases have mannan endo-1,4-beta-mannosidase activity (EC 3.2.1.78) that catalyzes the hydrolysis of 1,4-beta-D-mannosidic linkages in mannans, galactomannans and glucomannans. According to CAZy (www.cazy.org), endo-1,4-beta-mannanases have been found in glycoside hydrolyase families 5, 26 and 113. Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S).

### Proteases

Proteases are enzymes that hydrolyses peptide bonds. It includes any enzyme belonging to the EC 3.4 enzyme group (including each of the thirteen subclasses thereof (http://en.wikipedia.org/wiki/Category:EC_3.4). The EC number refers to Enzyme Nomenclature 1992 from NC-IUBMB, Academic Press, San Diego, California, including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The term “subtilases” refer to a sub-group of serine protease according to Siezen et al., Protein Eng. 4 (1991) 719-737 and Siezen et al. Protein Science 6 (1997) 501-523. Serine proteases or serine peptidases is a subgroup of proteases characterized by having a serine in the active site, which forms a covalent adduct with the substrate. Further, the subtilases (and the serine proteases) are characterized by having two active site amino acid residues apart from the serine, namely a histidine and an aspartic acid residue. The subtilases may be divided into six sub-divisions, i.e. the Subtilisin family, the Thermitase family, the Proteinase K family, the Lantibiotic peptidase family, the Kexin family and the Pyrolysin family. The term “protease activity” means a proteolytic activity (EC 3.4).

Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families.

Non-limiting examples of proteases that will be useful in combination with the endoglucanases of the invention are proteases selected from the group of proteases having SEQ ID NO: 402, SEQ ID NO: 406, SEQ ID NO: 407, SEQ ID NO: 408, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, and SEQ ID NO: 438.

### Lipases and Cutinases:

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147). Further, lipases from Geotrichum candidum as disclosed in WO2022/162043 may be useful.

### Amylases:

Suitable amylases which can be used together with the endoglucanases of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

### Peroxidases/Oxidases:

A peroxidase is a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity. Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

### Nucleases

Suitable nucleases include deoxyribonucleases (DNases) and ribonucleases (RNases) which are any enzyme that catalyzes the hydrolytic cleavage of phosphodiester linkages in the DNA or RNA backbone respectively, thus degrading DNA and RNA. Preferred DNases may be selected from any of the enzyme classes E.C. 3.1.21.X, where X =1, 2, 3, 4, 5, 6, 7, 8 or 9.

The DNase polypeptide is typically a microbial enzyme, preferably of fungal or bacterial origin, or a genetically engineered variant of a microbial DNase.

### Licheninases

Suitable licheninases (lichenases) include enzymes that catalyse the hydrolysis of the beta-1,4-glucosidic bonds to give beta-glucans. Licheninases (or lichenases) (e.g. EC 3.2.1.73) hydrolyse (1,4)-beta-D-glucosidic linkages in beta-D-glucans containing (1,3)- and (1,4)-bonds and can act on lichenin and cereal beta-D-glucans.

### Xanthanases

Xanthan gum is a natural polysaccharide consisting of different sugars which are connected by several different bonds, such as β-D-mannosyl-β-D-1,4-glucuronosyl bonds and β-D-glucosyl-β-D-1,4-glucosyl bonds. Xanthan gum is at least partly soluble in water and forms highly viscous solutions or gels. Complete enzymatic degradation of xanthan gum requires several enzymatic activities including xanthan lyase activity and endo-beta-1,4-glucanase activity, preferably a GH9 endoglucanase. Xanthan lyases are enzymes that cleave the β-D-mannosyl-β-D-1,4-glucuronosyl bond of xanthan, whereas the GH9 endoglucanase catalyses the hydrolysis of the glycosyl bond to release smaller sugars.

### Enzyme Formulations

### Liquid Enzyme Formulations

The enzyme of the present invention may be formulated as a liquid enzyme formulation, which is generally a pourable composition, though it may also have a high viscosity. The physical appearance and properties of a liquid enzyme formulation may vary a lot—for example, they may have different viscosities (gel to water-like), be colored, not colored, clear, hazy, and even with solid particles like in slurries and suspensions. The minimum ingredients are the enzyme of the present invention and a solvent system to make it a liquid. In addition to the enzyme of the present invention, the liquid enzyme formulation may also comprise other enzyme activities, such as protease, amylase, lipase, cellulase, and/or nuclease (e.g., DNase, RNase) activities.

The solvent system may comprise water, polyols (such as glycerol, (mono, di, or tri) propylene glycol, (mono, di, or tri) ethylene glycol, sugar alcohol (e.g. sorbitol, mannitol, erythritol, dulcitol, inositol, xylitol or adonitol), polypropylene glycol, and/or polyethylene glycol), ethanol, sugars, and salts. Usually the solvent system also includes a preservation agent and/or other stabilizing agents.

A liquid enzyme formulation may be prepared by mixing a solvent system and an enzyme concentrate with a desired degree of purity (or enzyme particles to obtain a slurry/suspension).

In an embodiment, the liquid enzyme composition comprises:
(a) at least 0.01% w/w active enzyme protein,
(b) at least 0.5% w/w polyol,
(c) water, and
(d) optionally a preservation agent.

The enzyme of the present invention in the liquid composition of the invention may be stabilized using conventional stabilizing agents. Examples of stabilizing agents include, but are not limited to, sugars like glucose, fructose, sucrose, or trehalose; addition of salt to increase the ionic strength; divalent cations (e.g., Ca²⁺ or Mg²⁺); and enzyme inhibitors, enzyme substrates, or various polymers (e.g., PVP). Selecting the optimal pH for the formulation may be very important for enzyme stability. The optimal pH depends on the specific enzyme but is typically in the range of pH 4-9. In some cases, surfactants like nonionic surfactant (e.g., alcohol ethoxylates) can improve the physical stability of the enzyme formulations.

One embodiment of the invention relates to a composition comprising a enzyme of the present invention, wherein the composition further comprises:
(i) a polyol, preferably selected from glycerol, (mono, di, or tri) propylene glycol, (mono, di, or tri) ethylene glycol, polyethylene glycol, sugar alcohols, sorbitol, mannitol, erythritol, dulcitol, inositol, xylitol and adonitol;
(ii) optionally an additional enzyme, preferably selected from protease, amylase, or lipase,
(iii) optionally a surfactant, preferably selected from anionic and nonionic surfactants,
(iv) optionally a divalent cation, polymer, or enzyme inhibitor.
(v) optionally having a pH in the range of pH 4-9; and
(vi) water.

Slurries or dispersions of enzymes are typically prepared by dispersing small particles of enzymes (e.g., spray-dried particles) in a liquid medium in which the enzyme is sparingly soluble, e.g., a liquid nonionic surfactant or a liquid polyethylene glycol. Powder can also be added to aqueous systems in an amount so not all go into solution (above the solubility limit). Another format is crystal suspensions which can also be aqueous liquids (see for example WO2019/002356). Another way to prepare such dispersion is by preparing water-in-oil emulsions, where the enzyme is in the water phase, and evaporate the water from the droplets. Such slurries/suspension can be physically stabilized (to reduce or avoid sedimentation) by addition of rheology modifiers, such as fumed silica or xanthan gum, typically to get a shear thinning rheology.

### Granular Enzyme Formulations

The enzyme of the present invention may also be formulated as a solid/granular enzyme formulation. Non-dusting granulates may be produced, e.g. as disclosed in U.S. Pat. Nos. 4, 106,991 and 4,661,452, and may optionally be coated by methods known in the art. Examples of waxy coating materials are poly (ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The enzyme of the present invention may be formulated as a granule for example as a co-granule that combines one or more enzymes or benefit agents (such as MnTACN or other bleaching components). Examples of such additional enzymes include proteases, amylases, lipases, cellulases, and/or nucleases (e.g., DNase, RNase). Each enzyme will then be present in more granules securing a more uniform distribution of enzymes in the detergent. This also reduces the physical segregation of different enzymes due to different particle sizes. Methods for producing multi-enzyme co-granulate for the detergent industry are disclosed in the IP.com disclosure IP-COM000200739D.

An embodiment of the invention relates to an enzyme granule/particle comprising a enzyme of the present invention. The granule is composed of a core, and optionally one or more coatings (outer layers) surrounding the core. Typically, the granule/particle size, measured as equivalent spherical diameter (volume based average particle size), of the granule is 20-2000 μm, particularly 50-1500 μm, 100-1500 μm or 250-1200 μm.

The core may include additional materials such as fillers, fibre materials (cellulose or synthetic fibres), stabilizing agents, solubilising agents, suspension agents, viscosity regulating agents, light spheres, plasticizers, salts, lubricants and fragrances. The core may include binders, such as synthetic polymer, wax, fat, or carbohydrate. The core may comprise a salt of a multivalent cation, a reducing agent, an antioxidant, a peroxide decomposing catalyst and/or an acidic buffer component, typically as a homogenous blend. The core may consist of an inert particle with the enzyme absorbed into it, or applied onto the surface, e.g., by fluid bed coating. The core may have a diameter of 20-2000 μm, particularly 50-1500 μm, 100-1500 μm or 250-1200 μm. The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation. Methods for preparing the core can be found in Hand-book of Powder Technology; Particle size enlargement by C. E. Capes; Volume 1; 1980; Elsevier. These methods are well-known in the art and have also been described in international patent application WO2015/028567, pages 3-5, which is incorporated by reference.

The core of the enzyme granule/particle may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA). Examples of enzyme granules with multiple coatings are shown in WO 93/07263 and WO 97/23606.

Such coatings are well-known in the art, and have earlier been described in, for example, WO00/01793, WO2001/025412, and WO2015/028567, which are incorporated by reference.

In one aspect, the present invention provides a granule, which comprises:
(a) a core comprising a enzyme of the present invention according to the invention; and
(b) optionally a (salt) coating consisting of one or more layer(s) surrounding the core.

Another aspect of the invention relates to a layered granule, comprising:
(a) a (non-enzymatic) core;
(b) a coating surrounding the core, wherein the coating comprises a enzyme of the present invention; and
(c) optionally a (salt) coating consisting of one or more layer(s) surrounding the enzyme containing coating.

### Encapsulated Enzyme Formulations

The enzyme of the present invention may also be formulated as an encapsulated enzyme formulation (an ‘encapsulate’). This is particularly useful for separating the enzyme from other ingredients when the enzyme is added into, for example, a (liquid) cleaning composition, such as the detergent compositions described below.

Physical separation can be used to solve incompatibility between the enzyme(s) and other components. Incompatibility can arise if the other components are either reactive against the enzyme, or if the other components are substrates of the enzyme. Other enzymes can be substrates of proteases.

The enzyme may be encapsulated in a matrix, preferably a water-soluble or water dispersible matrix (e.g., water-soluble polymer particles), for example as described in WO 2016/023685. An example of a water-soluble polymeric matrix is a matrix composition comprising polyvinyl alcohol. Such compositions are also used for encapsulating detergent compositions in unit-dose formats. The enzyme may also be encapsulated in core-shell microcapsules, for example as described in WO 2015/144784, or as described in the IP.com disclosure IPCOM000239419D.

Such core-shell capsules can be prepared using a number of technologies known in the art, e.g., by interfacial polymerization using either a water-in-oil or an oil-in-water emulsion, where polymers are crosslinked at the surface of the droplets in the emulsion (the interface between water and oil), thus forming a wall/membrane around each droplet/capsule.

Unit-dose detergents is a common terminology covering detergents delivered in single-use format; these can be pouches of poly(vinyl alcohol) containing liquid detergents or powder detergents in single or multiple chambers with either all liquid chambers or hybrid models with both liquid and powder chambers. Unit-dose detergents can also be pressed tablets with one or more layers (‘phases’). Laundry unit-dose liquid compositions are generally low in water (<10% w/w);

high in surfactants (50 to 65%), high in solvents (15 to 30% polyols) relative to typical mass fraction levels in aqueous HDL.

### Purity of Enzyme in Formulations

The enzyme of the present invention used in the above-mentioned enzyme formulations may be purified to any desired degree of purity. This includes high levels of purification, as achieved for example by using methods of crystallization-but also none or low levels of purification, as achieved for example by using crude fermentation broth, as described in WO 2001/025411, or in WO 2009/152176.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids and, or slurries.

Non-dusting granulates may be produced, e.g. as disclosed in U.S. Pat. Nos. 4, 106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water-soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water-soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water-soluble film. The compartment for liquid components can be different in composition than compartments containing solids. Ref: (US2009/0011970 A1).

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent. A liquid or gel detergent may be non-aqueous.

### Use in Detergents

The polypeptides of the present invention may be added to and thus become a component of a detergent composition.

The detergent composition of the present invention may be formulated, for example, as a hand or machine laundry detergent composition including a laundry additive composition suitable for pretreatment of stained fabrics or for rejuvenating textile (e.g. by fuzz or pill removal) to restore some of the visual and feel properties of fabrics after extended use to match that of a new textile, and a rinse added fabric softener composition, or be formulated as a detergent composition for use in general household hard surface cleaning operations, or be formulated for hand or machine dishwashing operations.

Further, in WO 2022/184568 it is disclosed that the addition of cellulase enzymes to consumer products can improve the deposition of fragrance on to textiles. This use of the polypeptides of the present invention is also encompassed.

In a specific aspect, the present invention provides a detergent additive comprising a polypeptide of the present invention as described herein.

### Embodiments

The following embodiments are part of the disclosed invention:

1. A polypeptide having endoglucanase activity, characterized in that said polypeptide consists of a catalytic domain, a linker, and a carbohydrate binding module (CBM), wherein the linker is of the form (P/X)aG wherein the value of a is in the range 8-16, X is selected from the amino acids G, V, N, F and D, and (P/X)a specifies that each position in the linker is selected from the group consisting of the amino acids G, V, N, F, D and P, and wherein further the linker comprises at least one and not more than five amino acids selected from the group consisting of G, V, N, F and D.

2. The polypeptide of embodiment 1, characterized in that the catalytic domain has a least 80% sequence identity to SEQ ID NO:394, the CBM has at least 80% sequence identity to SEQ ID NO:395 or to SEQ ID NO:396.

3. The polypeptide of claim 1 or 2, wherein each of the G, V, N, F and D when present in the linker are separated by at least one P, such as 1, 2, 3, 4, 5, 6, 7, 8, 9, 10, 11, 12, 13, 14 or 15 P.

4. The polypeptide according to any of embodiments 1 to 3, wherein the at least the first amino acid residue in the linker, such as the first 1, 2, 3, 4, 5, 6, 7, 8, 9, 10, 11, 12, 13, 14 or 15 amino acid residues, is P.

5. The polypeptide according to any of embodiments 1 to 4, wherein
a. X is a glycine residue (G), so that (P/X)ₐ specifies that each position in the linker is selected from the group of amino acids consisting of P and G,
b. the value of a is in the range 10-16, preferably 13 to 16, preferably 13
c. the linker comprises 2, 3, 4 or 5 G, preferably 2, 3 or 4, preferably 2 or 3 G,
d. each of the glycine residues are separated by at least one P, such as 1, 2, 3, 4, 5, 6, 7, 8, 9, 10, 11, 12, 13 or 14 P, and
e. wherein the linker further has at least one P before the first G in the linker, such 1, 2, 3, 4, 5, 6, 7, 8, 9, 10, 11, 12, 13 or 14 P before the first G in the linker.

6. The polypeptide according to any of embodiments 1 to 5, wherein the catalytic domain has at least 80% sequence identity to SEQ ID NO:394, such as at least 85%, 90%, 91%, 92%, 93%, 94%, 95%, 96%, 97%, 98%, 99% or even 100% sequence identity to SEQ ID NO: 394.

7. The polypeptide according to any of embodiments 1 to 5, wherein the CBM has at least 80%, such as 85%, 90%, 91%, such as 92%, 93%, 94%, 95%, 96%, 97%, 98%, 99% or even 100% sequence identity to SEQ ID NO:395 or to SEQ ID NO:396.

8. The polypeptide according to any of embodiments 1 to 7, wherein the linker is selected from the group consisting of SEQ ID NO:26, SEQ ID NO:97, SEQ ID NO:93 and SEQ ID NO: 404.

9. The polypeptide according to any of embodiments 1 to 8, wherein the polypeptide is selected from the group of polypeptides having at least 80% sequence identity, such as 85%, 90%, 91%, 92%, 93%, 94%, 95%, 96%, 97%, 98%, 99% or even 100% sequence identity to any of SEQ ID NO:397, SEQ ID NO:398, SEQ ID NO:399, SEQ ID NO:400, and SEQ ID NO:401.

10. The polypeptide of any of the preceding embodiments, which is a family GH45 endoglucanase.

11. The polypeptide of any of the preceding embodiments, wherein the CBM is a CBM1.

12. The polypeptide of any of the preceding embodiments, wherein the polypeptide comprises an N-terminal catalytic domain and a C-terminal CBM.

13. The polypeptide of any of the preceding embodiments, wherein the polypeptide comprises a C-terminal catalytic domain and a N-terminal CBM.

14. The polypeptide according to any of the preceding embodiments, wherein the linker stability in an aqueous composition comprising a protease as determined by the Linker Stabililtiy Assay (Example 2) is at least 0.1, or 0.15 or 0.2.

15. The polypeptide according to any of embodiments 1 to 14, wherein the wash performance as determined by the Full Scale Washing assay (Example 7) is at least 80% of the wash performance of the reference enzyme having SEQ ID NO:391, such as at least 85%, 90%, 91%, 92%, 93%, 94%, 94%, 95%, 96%, 97%, 98%, 99%, 100% or even superior wash performance of the reference enzyme having SEQ ID NO:391.

16. The polypeptide of any of embodiment 1 to 15, wherein the polypeptide demonstrates improved fabric or textile care and/or improved wash performance relative to the endoglucanase having SEQ ID NO:391 after storage in the presence of protease.

17. The polypeptide according to any of embodiments 1 to 13, wherein the wash performance of the polypeptide before incubation with the protease having SEQ ID NO:402 is at least 80%, such as 85%, 90%, 91%, 92%, 93%, 94%, 95%, 96%, 97%, 98%, 99% or even at least 100% of the wash performance of reference polypeptide having SEQ ID NO: 391 before incubation with the protease having SEQ ID NO:402, wherein the wash performance of the polypeptides before incubation with the protease having SEQ ID NO: 402 is determined by the method described in Example 7 (Full Scale Washing).

18. In an embodiment of embodiment 17 the wash performance of the polypeptide is improved compared to the wash performance of reference polypeptide having SEQ ID NO: 391 before incubation with the protease having SEQ ID NO:402.

19. The polypeptide according to any of embodiments 1 to 13, wherein the wash performance of the polypeptide in the presence of a protease before incubation with the protease having SEQ ID NO:402 is at least 80%, such as 85%, 90%, 91%, 92%, 93%, 94%, 95%, 96%, 97%, 98%, 99% or even 100% of the wash performance of reference polypeptide having SEQ ID NO:391 before incubation with the protease having SEQ ID NO: 402, wherein the wash performance of the polypeptides before incubation with the protease having SEQ ID NO:402 is determined by the described in Example 7.

20. A composition comprising one or more of the polypeptides of embodiments 1 to 19, at least one detergent component and optionally one or more additional enzymes.

21. The composition according to embodiment 20, wherein the one or more additional enzymes is selected from the group consisting of amylases, proteases, peroxidases, cellulases, betaglucanases, xyloglucanases, hemicellulases, xanthanases, xanthan lyases, lipases, acyl transferases, phospholipases, esterases, laccases, catalases, aryl esterases, amylases, alpha-amylases, glucoamylases, cutinases, pectinases, pectate lyases, keratinases, reductases, oxidases, phenoloxidases, lipoxygenases, ligninases, carrageenases, pullulanases, tannases, arabinosidases, hyaluronidases, chondroitinases, xyloglucanases, xylanases, pectin acetyl esterases, polygalacturonases, rhamnogalacturonases, other endo-beta-mannanases, exo-beta-mannanases (GH5 and/or GH26), licheninases, phosphodiesterases, pectin methylesterases, cellobiohydrolases, transglutaminases, nucleases, and combinations thereof.

22. The composition according to any of embodiments 20 or 21, wherein the composition comprises a protease.

23. The composition according to any of embodiments 20 to 22, being a detergent composition.

24. The composition according to any of embodiments 20 to 23, wherein the at least one detergent component is selected from the group consisting of surfactants, hydrotropes, builders, co-builders, chelators or chelating agents, bleaching system or bleach components, polymers, fabric hueing agents, fabric conditioners, foam boosters, suds suppressors, dispersants, dye transfer inhibitors, fluorescent whitening agents, perfume, tannish inhibitors, optical brighteners, bactericides, fungicides, soil suspending agents, soil release polymers, anti-redeposition agents, enzyme inhibitors or stabilizers, enzyme activators, antioxidants, and solubilizers, and combinations thereof.

25. The composition according to any of embodiments 20 to 24, being a liquid detergent composition.

26. Use of a polypeptide according to any of embodiments 1 to 19 or a composition according to any of embodiments 20 to 25 for cleaning fabric, textiles or hard surfaces.

27. Use of a polypeptide according to any of embodiments 1 to 19 or a composition according to any of embodiments 20 to 25 for fabric or textile care, such as for pre-treatment of stained fabrics or for rejuvenating textile (e.g. by fuzz or pill removal), to restore the visual and feel properties of fabrics after extended use to match that of a new textile.

28. A method for washing an object, such as a fabric or textile, comprising
a. Providing a wash liquor by dissolving/mixing a polypeptide according to any of embodiments 1 to 19 or a composition according to any of embodiments 20 to 25 in water to provide a wash liquor
b. washing the object in the wash liquor
c. draining the wash liquor and optionally repeating the wash cycle; and
d. rinsing and optionally drying the object.

29. Use of a polymer of any of embodiments 1 to 19 for replacing one or more polymers partly or fully in a composition according to any of embodiments 20 to 25.

30. A polynucleotide encoding the polypeptide of any of embodiments 1 to 19.

31. The polynucleotide according to embodiment 30 being an isolated polynucleotide.

32. A nucleic acid construct or expression vector comprising the polynucleotide of any of embodiment 30 or 31.

33. A recombinant host cell transformed with the polynucleotide of any of embodiments 30 or 31.

34. A method of producing a polypeptide, comprising:
a. cultivating the recombinant host cell of embodiment 33 under conditions suitable for expression of the variant; and
b. recovering the polypeptide.

35. A method for cleaning a surface comprising contacting the surface with a polypeptide of any of embodiments 1 to 19 or a composition of any of embodiments 20 to 25.

36. A method for hydrolyzing a cellulosic material partly or fully comprising adding to said material a polypeptide of any of embodiments 1 to 19.

### EXAMPLES

### Materials and Methods

General methods of PCR, cloning, ligation nucleotides etc. are well-known to a person skilled in the art and may for example be found in in “Molecular cloning: A laboratory manual”, Sambrook et al. (1989), Cold Spring Harbor lab., Cold Spring Harbor, NY; Ausubel, F. M. et al. (eds.); “Current protocols in Molecular Biology”, John Wiley and Sons, (1995); Harwood, C. R., and Cutting, S. M. (eds.); “DNA Cloning: A Practical Approach, Volumes I and II”, D. N. Glover ed. (1985); “Oligonucleotide Synthesis”, M. J. Gait ed. (1984); “Nucleic Acid Hybridization”, B. D. Hames & S. J. Higgins eds (1985); “A Practical Guide To Molecular Cloning”, B. Perbal, (1984).

TABLE A
Composition of Model detergent A

| | Active content |
|---|---|
| Ingredient | (Wt %) |
| LAS (linear alkylbenzene-sulfonic acid) | 12% |
| AEOS/SLES (alcohol ether sulfate/sodium | 4% |
| lauryl ether sulfate) | |
| AEO (alcohol ethoxylate) | 12% |
| Soap | 3% |
| Sodium citrate | 3.9% |
| Diethylenetriaminepentakis(methylene)penta- | 1.5% |
| kis(phosphonic acid), heptasodium salt (DTPMP Na7) | |
| TEA (triethanolamine) | 2% |
| MPG (monopropylene glycol) | 2% |
| Ethanol | 3% |
| Phenoxyethanol | 0.5% |
| NaOH | 0.5% |
| Water | Ad 100% |

TABLE B
Composition Model Detergent B

| | Active content |
|---|---|
| Ingredient | (Wt %) |
| Sodium linear alkylbenzene sulfonate (Na-LAS) | 12% |
| Sodium lauryl ether sulfate (SLES) | 4.1% |
| Alcohol ethoxylate with 7 mol EO (AEO) | 12% |
| Topped Palm Kernel Fatty Acid | 3.0% |
| Sodium hydroxide | 0.5% |
| Ethanol | 6.0% |
| Propylene glycol (MPG) | 2.0% |
| Triethanolamine (TEA) | 2.0% |
| Trisodium citrate dihydrate | 3.9% |
| Diethylenetriaminepentakis(methylene)penta- | 1.6% |
| kis(phosphonic acid), heptasodium salt (DTPMP Na7) | |
| 2-Phenoxyethanol | 0.5% |
| Water (demineralized) | Ad 100% |
| pH target 8.2-8.6 | 8.4 |

TABLE C
Composition Model Detergent C

| | Active content |
|---|---|
| Ingredient | (Wt %) |
| Llinear alkylbenzene sulfonic acid (LAS acid) | 22% |
| Alcohol ethoxylate with 7 mol EO (AEO-7) | 26% |
| Topped Palm Kernel Fatty Acid | 9.4% |
| Monoethanol amine (MEA) | 7.1% |
| Glycerol | 8.4% |
| Propylene glycol (MPG) | 15.7% |
| Diethylenetriaminepentakis(methylene)pentakis(phos- | 0.5% |
| phonic acid), heptasodium salt (DTPMP Na7) | |
| Water (demineralized) | To 100% |
| pH target 7.5-8.0 | 8.0 |

### Protease

The proteases used for Linker Stability Assay is the commercially available protease having SEQ ID NO: 402.

The protease used for Full Scale Washing is the commercially available protease having SEQ ID NO: 406.

The proteases used for co-granulation (Example 8) is the commercially available protease having SEQ ID NO: 407.

The proteases used for comparison of stability of cellulase of the present invention with cellulase from Acremonium thermophilum (Example 9) is the commercially available protease having SEQ ID NO: 408.

### Reference Endoglucanase

The endoglucanase used as a reference is a commercially available endoglucanase having SEQ ID NO: 391.

### Representation of Endoglucanase Variants in the Tables

The endoglucanase variants in the tables in this section are represented in the form Catalytic domain (N-terminal)-Linker-CBM (C-terminal), e.g. from Table 2:

| Catalytic | | | | |
|---|---|---|---|---|
| domain | | CBM | Linker | |
| (N- | | (C- | stabil- | Linker |
| terminal) | Linker | terminal) | ity | sequence |
| SEQ ID NO: | SEQ ID | SEQ ID NO: | 0.21 | GPPPPPPPPPPPPG |
| 394 | NO: 1 | 395 | | |

Excerpt from Table 2: Representation of Endoglucanases

Thus, the endoglucanase variant represented in Table 2 as above could equally be represented as (SEQ ID NO:403):

ASGSGQSTRY WDCCKPSCAW PGKAAVSQPV YSCDANFQRL
SDFNVQSGCN GGSAYACADQ TPWAVNDNLA YGFAATSIAG
GSESSWCCAC YALTFTSGPV AGKTMVVQST STGGDLGSNH
FDIAMPGGGV GIFDGCSSQF GGLPGDRYGG ISSRDQCDSF
PAPLKPGCYW RFDWFQNADN PTVTFQQVQC PAEIVARSGC
KRNDDSSFPV FTGPPPPPPP PPPPPGCTTQ KWGQCGGIGY
TGCTNCVAGT TCTQLNPWYH QCR

The linker sequence is additionally set forth in the righthand column for ease of reference.

### Example 1: Construction of Variants

Endoglucanase variants were constructed of the Thielavia terrestris endoglucanase (SEQ ID NO: 391). The variants were made by traditional cloning of DNA fragments (Sambrook et al., Molecular Cloning: A Laboratory Manual, 2nd Ed., Cold Spring Harbor, 1989) using PCR together with properly designed oligonucleotides that introduced the desired mutations in the resulting sequence. Alternatively, synthetic gene fragments purchased from vendors such IDTDNA were used to replace the native DNA sequence with the new, desired DNA sequence.

The oligos are designed corresponding to the DNA sequence flanking the desired site(s) of mutation or stretch of DNA to be replaced, separated by the DNA base pairs defining the insertions/deletions/substitution/synthetic DNA sequence, and purchased from an oligo vendor such as IDTDNA. In order to test the endoglucanases of the invention, the mutated DNA comprising a endoglucanase of the invention are integrated into a competent A. oryzae strain by homologous recombination, fermented using standard protocols (yeast extract based media, 4-5 days, 30° C.), and purified as follows.

Culture broth is filtered through a Nalgene 0.2 μm filtration unit to remove the Aspergillus host cells. The pH in the filtrate is adjusted to pH 4.0 with 20% CH₃COOH and the pH adjusted filtrate was applied to a Capto MMC column (from GE Healthcare) equilibrated in 20 mM CH₃COOH/NaOH, 1 mM CaCl₂, pH 4.0. After washing the column extensively with the equilibration buffer, the endoglucanase is eluted with 50 mM Tris-base, 1 mM CaCl₂, unbuffered. Fractions from the column are analysed for endoglucanase activity. The endoglucanase peak is pooled and applied to a Q-sepharose FF column (from GE Healthcare) equilibrated in 50 mM Tris/HCl, pH 9.0. After washing the column extensively with the equilibration buffer, the endoglucanase is eluted with a linear NaCl gradient over three column volumes between the equilibration buffer and 50 mM Tris/HCl, 5 mM CaCl₂, 500 mM NaCl, pH 9.0. Fractions from the column are analyzed for endoglucanase activity and the endoglucanase peak is pooled as the purified product. The purified endoglucanases are analyzed by SDS-PAGE. As the endoglucanase variants are glycosylated they gave diffuse bands on coomassie stained SDS-PAGE gels. The purified products are used for further characterization.

### Example 2: Linker Stability Assay

### Principle

The linker stability is measured by (A) incubating the endoglucanase in detergent containing protease, then (B) determining the ability of the incubated endoglucanase to bind to cellulose fibers. If the linker or the cellulose binding domain is affected by the protease the binding affinity of the endoglucanase to cellulose fibers will be reduced.

The binding is determined by adding a dilution of the incubated endoglucanase to a suspension of cellulose fibers. After incubation at 5° C., the endoglucanase bound to cellulose is removed by centrifugation, and the amount of endoglucanase not bound to the cellulose is determined by measuring (C) the activity of endoglucanase in the supernatant. The activity of the endoglucanase not bound to the cellulose relative to the activity of a parallel sample incubated at similar conditions but in the absence of cellulose is a measure of the linker stability.

The activity is based on hydrolysis of the soluble carboxymethyl cellulose (CMC) followed by (D) detection of the number of reducing ends formed. CMC is a substrate both for the intact endoglucanase and endoglucanases having no cellulose binding domain.

### A. Incubation in Detergent Containing Protease

### Chemicals

Detergent: Model Detergent A
Protease: SEQ ID NO: 402
HEPES: 4-(2-hydroxyethyl)-1-piperazineethanesulfonic acid, Sigma H3375

### Reagents

Dilution Buffer: 50 mM HEPES, pH 8
Protease stock solution, protease of SEQ ID NO: 402
Detergent with 0.3 mg/mL Protease: 300 ppm in Model Detergent A

### Procedure

(1) Detergent with 0.3 mg/mL protease is prepared by adding Protease stock solution to 100 mL Detergent to a final protease concentration of 300 ppm active protease protein in the detergent and mix for 1 hour by magnetic stirring at room temperature.
(2) The endoglucanases are diluted to 300 ppm in Dilution Buffer.
(3) 270 μL of Detergent with Protease from (1) is pipetted into 96-well polypropylene micro-plates (Thermo Scientific™ 249944) in well positions A1 to D12.
(4) 30 μL of diluted endoglucanase from (2) is added to each well (positions A1 to D12). Each endoglucanase is tested in triplicates and positions D4 to D6 are used for blanks, where 30 μL Milli Q water is added instead of endoglucanase.
(5) Small magnets are added to each well (position A1 to D12) and the plate is sealed with heat seal (Thermo Scientific™ Adhesive PCR Plate Seals AB0558) followed by mixing by magnetic stirring for 30 minutes.
(6) After mixing, the plate is incubated at the time and temperature indicated in the examples.

### B. Binding to Cellulose Fibers

### Chemicals

Cellulose fibers: Avicel®, PH-101, (Sigma 11365)
HEPES: 4-(2-hydroxyethyl)-1-piperazineethanesulfonic acid, Sigma H3375

### Reagents

Binding Buffer: 50 mM HEPES, pH 8
Avicel suspension: 1.25 g/100 mL Avicel in Binding Buffer, mixed 1 hour before use

### Procedure

1) 180 μL Avicel suspension is added to position A1 to D12 in new 96-well microplates (Thermo Scientific™ cat. No. 269620) and 180 μL Binding Buffer was added to position E1 to H12
2) 20 μL sample aliquots from each well in the incubated plate from step A is then added to the wells in position A1 to D12 and in position E1 to H12, respectively.
3) The plate is shaken at a speed sufficient for keeping the cellulose fibers in suspension for 1 hour at 5° C. to allow the endoglucanase to bind to cellulose
4) After binding, the plate is centrifuged for 10 sec. at 1500 rpm and the supernatant diluted 2.5-fold in Binding Buffer (40 μL sample +60 μL buffer). Both supernatants from Avicel and corresponding wells without Avicel are diluted.

### C. CMC Activity Assay

### Chemicals

CMC: Sodium carboxymethyl cellulose (Sigma C5678)
K-Na-Tartrate: Merck 8087
Beta-glucosidase Megazyme (Thermotoga maritima; accession number Q08638, Megazyme Cat. No. E-BGOS™) diluted to 0.1 mg/mL
PAHBAH 4-Hydroxybenzhydrazide (Sigma H9882)
NaOH sodium hydroxide (J.T. Baker 0402.1000)
HEPES: 4-(2-hydroxyethyl)-1-piperazineethanesulfonic acid, Sigma H3375

### Reagents

Assay Buffer: 50 mM HEPES, pH 8
CMC Substrate: 1.25 g CMC/100 mL Assay Buffer, mixed 1 hr before use
PAHBAH Buffer: 50 g/L K-Na-tartrate+20 g/L NaOH
PAHBAH Reagent: 15 mg/mL PAHBAH in PAHBAH Buffer
Beta-glucosidase Solution: 0.1 mg/mL beta-glucosidase in Assay Buffer

### Procedure

1) 160 μL CMC Substrate is pipetted into new 96-well microplates (Thermo Scientific™ cat. No. 269620)
2) 20 μL diluted supernatant from step B is added together with 20 μL beta-glucosidase Solution
3) The plate was sealed with heat seal (Thermo Scientific™ Adhesive PCR Plate Seals AB0558), and incubated for 45 minutes at 40° C.
4) After reaction, 100 μL from each well is transferred to ThermoFast 96 non-skirted® PCR plate (Thermo Scientific™ cat. No. AB-0600) followed by 75 μL PAHBAH Reagent
5) The plate from (4) is then sealed with sealing foil (Greiner bio-one plate sealer, Cat. No. 676001) and incubated at 95° C. for 10 minutes followed by cooling at 10° C. for 5 minutes in BioRad T100™ Thermal cycler PCR machine
6) After cooling, 100 μL aliquots are transferred to new 96-well microplates (Thermo Scientific™ cat. No. 269620) and the absorbance is read at 405 nm (A405nm). The absorbance is an expression of the activity of the endoglucanase in the supernatant.

### D. Data-Treatment

1) From the absorbance readings from step C, the average of the 3 blanks from the wells without Avicel, A405nm (blank_ref), is calculated (position H4 to H6), and the average of the 3 blanks from the wells with Avicel, A405nm (blank_Avicel) is calculated (position D4 to D6).
2) The absorbance readings from the endoglucanase containing wells are then corrected for their respective blanks (i.e. those from (1)).
3) The linker stability is calculated as

$$1 - [ Act 405 nm ( + Avicel ) / Act 405 nm ( - Avicel ) ] ,$$

where Act405nm (+Avicel) and Act405nm (−Avicel) is the activity (i.e. absorbance corrected for blank) in the well with supernatant from incubation with Avicel and without Avicel, respective-ly.

The linker stabilities reported in the examples are the averages of the triplicates analyzed.

### Example 3: Stability of Endoglucanases with P-Linkers

Endoglucanases were prepared as described in Example 1. The stability was determined using the assay described in Example 2 (Linker Stability Assay), where the stress condition was incubation at 45° C. for 5 days in detergent with the protease of SEQ ID NO:402 before analyzing the residual activity. Results are shown in Table 1.

TABLE 1
Linker stability for proline-rich linkers comprising 7 to 20 proline residues.

| Catalytic domain | | CBM | Linker | |
|---|---|---|---|---|
| (N-terminal) | Linker | (C-terminal) | stability | Linker sequence |
| Reference endoglucanase SEQ ID NO: 391 | | | 0.00 | |
| SEQ ID NO: 394 | SEQ ID | SEQ ID NO: 395 | 0.25 | PPPPPPPG |
| | NO: 378 | | | |
| SEQ ID NO: 394 | SEQ ID | SEQ ID NO: 395 | 0.48 | PPPPPPPPG |
| | NO: 379 | | | |
| SEQ ID NO: 394 | SEQ ID | SEQ ID NO: 395 | 0.46 | PPPPPPPPPG |
| | NO: 380 | | | |
| SEQ ID NO: 394 | SEQ ID | SEQ ID NO: 395 | 0.51 | PPPPPPPPPPG |
| | NO: 381 | | | |
| SEQ ID NO: 394 | SEQ ID | SEQ ID NO: 395 | 0.35 | PPPPPPPPPPPG |
| | NO: 382 | | | |
| SEQ ID NO: 394 | SEQ ID | SEQ ID NO: 395 | 0.41 | PPPPPPPPPPPPG |
| | NO: 383 | | | |
| SEQ ID NO: 394 | SEQ ID | SEQ ID NO: 395 | 0.52 | PPPPPPPPPPPPPG |
| | NO: 384 | | | |
| SEQ ID NO: 394 | SEQ ID | SEQ ID NO: 395 | 0.53 | PPPPPPPPPPPPPPG |
| | NO: 385 | | | |
| SEQ ID NO: 394 | SEQ ID | SEQ ID NO: 395 | 0.56 | PPPPPPPP |
| | NO: 386 | | | PPPPPPPG |
| SEQ ID NO: 394 | SEQ ID | SEQ ID NO: 395 | 0.45 | PPPPPPPP |
| | NO: 387 | | | PPPPPPPPG |
| SEQ ID NO: 394 | SEQ ID | SEQ ID NO: 395 | 0.52 | PPPPPPPP |
| | NO: 388 | | | PPPPPPPPPG |
| SEQ ID NO: 394 | SEQ ID | SEQ ID NO: 395 | 0.54 | PPPPPPPP |
| | NO: 389 | | | PPPPPPPPPP PPG |

Results clearly demonstrate that under these very stressed conditions, the stability of endoglucanases with different proline-rich linker lengths are much improved relative to the endoglucanase of SEQ ID NO:391.

### Example 4: Stability of Endoglucanases (P/G)₁₃G Linkers

Endoglucanases were prepared as described in Example 1. The stability was determined using the assay described in Example 2 (Linker Stability Assay), where the stress condition was incubation at 45° C. for 5 days with protease of SEQ ID NO:402 before analyzing the residual activity.

Results are shown in Table 2:

TABLE 2
Stability of linkers in endoglucanase variants of the invention

| Catalytic domain | | CBM | Linker | |
|---|---|---|---|---|
| (N-terminal) | Linker | (C-terminal) | stability | Linker sequence |
| SEQ ID NO: 391 | | | 0.00 | |
| SEQ ID NO: 394 | SEQ ID NO: 1 | SEQ ID NO: 395 | 0.21 | GPPPPPPPPPPPPG |
| SEQ ID NO: 394 | SEQ ID NO: 2 | SEQ ID NO: 395 | 0.34 | PGPPPPPPPPPPPG |
| SEQ ID NO: 394 | SEQ ID NO: 4 | SEQ ID NO: 395 | 0.40 | PPGPPPPPPPPPPG |
| SEQ ID NO: 394 | SEQ ID NO: 8 | SEQ ID NO: 395 | 0.46 | PPPGPPPPPPPPPG |
| SEQ ID NO: 394 | SEQ ID NO: 15 | SEQ ID NO: 395 | 0.53 | PPPPGPPPPPPPPG |
| SEQ ID NO: 394 | SEQ ID NO: 26 | SEQ ID NO: 395 | 0.24 | PPPPPGPPPPPPPG |
| SEQ ID NO: 394 | SEQ ID NO: 42 | SEQ ID NO: 395 | 0.47 | PPPPPPGPPPPPPG |
| SEQ ID NO: 394 | SEQ ID NO: 64 | SEQ ID NO: 395 | 0.55 | PPPPPPPGPPPPPG |
| SEQ ID NO: 394 | SEQ ID NO: 93 | SEQ ID NO: 395 | 0.53 | PPPPPPPPGPPPPG |
| SEQ ID NO: 394 | SEQ ID NO: 130 | SEQ ID NO: 395 | 0.24 | PPPPPPPPPGPPPG |
| SEQ ID NO: 394 | SEQ ID NO: 176 | SEQ ID NO: 395 | 0.57 | PPPPPPPPPPGPPG |
| SEQ ID NO: 394 | SEQ ID NO: 299 | SEQ ID NO: 395 | 0.51 | PPPPPPPPPPPPGG |

Results clearly demonstrate that under these very stressed conditions, the stability of endoglucanases with different proline-rich linker comprising glycine residues are much improved relative to the endoglucanase of SEQ ID NO:391.

### Example 5: Stability of Endoglucanases with (P/G)ₐG Linkers

Endoglucanases were prepared as described in Example 1. The stability was determined using the assay described in Example 2 (Linker Stability Assay), where the stress condition was incubation at 45° C. for 3 days with the protease of SEQ ID NO:402 before analyzing the residual activity. Results are shown in Table 3.

TABLE 3
Effect of different chain length and with one or two prolines substituted with glycine
residues at different locations. Results represents an average from two experiments

| Catalytic | | | | |
|---|---|---|---|---|
| domain | | CBM | | |
| (N-terminal) | Linker | (C-terminal) | Linker stability | Linker sequence |
| Reference endoglucanase SEQ ID NO: 391 | | | 0.00 | |
| SEQ ID | SEQ ID NO: | SEQ ID NO: 395 | 0.42 | PPPPPPPG |
| NO: 394 | 378 | | | |
| SEQ ID | SEQ ID NO: | SEQ ID NO: 395 | 0.71 | PPGPPPPPGPPPPG |
| NO: 394 | 97 | | | |
| SEQ ID | SEQ ID NO: | SEQ ID NO: 395 | 0.52 | PPPGPPPPPPPPPPG |
| NO: 394 | 410 | | | |
| SEQ ID | SEQ ID NO: | SEQ ID NO: 395 | 0.47 | PPGPPGPPPG |
| NO: 394 | 411 | | | |
| SEQ ID | SEQ ID NO: 8 | SEQ ID NO: 395 | 0.56 | PPPGPPPPPPPPPG |
| NO: 394 | | | | |
| SEQ ID | SEQ ID NO: | SEQ ID NO: 395 | 0.60 | PPPGPPPPG |
| NO: 394 | 412 | | | |
| SEQ ID | SEQ ID NO: | SEQ ID NO: 395 | 0.58 | PPPGPPPPPPG |
| NO: 394 | 413 | | | |
| SEQ ID | SEQ ID NO: | SEQ ID NO: 395 | 0.52 | PPPGPPPPPPPG |
| NO: 394 | 414 | | | |
| SEQ ID | SEQ ID NO: | SEQ ID NO: 395 | 0.66 | PPPGPPPPPG |
| NO: 394 | 415 | | | |
| SEQ ID | SEQ ID NO: | SEQ ID NO: 395 | 0.54 | PPGPPPPPPPPPG |
| NO: 394 | 416 | | | |
| SEQ ID | SEQ ID NO: | SEQ ID NO: 395 | 0.60 | PPPGPPPPPPPPPPPG |
| NO: 394 | 417 | | | |
| SEQ ID | SEQ ID NO: | SEQ ID NO: 395 | 0.61 | PPPGPPPPPPPPPPPPG |
| NO: 394 | 418 | | | |
| SEQ ID | SEQ ID NO: | SEQ ID NO: 395 | 0.46 | PPPGPPPPPPPPPPPPPG |
| NO: 394 | 419 | | | |
| SEQ ID | SEQ ID NO: | SEQ ID NO: 395 | 0.49 | PPGPPPPGPPPPPPPPPG |
| NO: 394 | 420 | | | |
| SEQ ID | SEQ ID NO: | SEQ ID NO: 395 | 0.75 | PPGGPPPPPPPPPPPPPG |
| NO: 394 | 421 | | | |

Results clearly demonstrate that replacing one or two of the prolines in the linker sequence by glycine residues are much improved relative to the endoglucanase of SEQ ID NO:391.

### Example 6: Stability of Endoglucanases with inserts of V, N, F or D

Endoglucanases were prepared as described in Example 1. The stability was determined using the assay described in Example 2 (Linker Stability Assay), where the stress condition was incubation at 45° C. for 3 days in detergent with the protease of SEQ ID NO:402 before analyzing the residual activity. Results are shown in Table 4.

TABLE 4
Effect of substituting proline with other amino acid residues.

| Catalytic domain | | | Linker | |
|---|---|---|---|---|
| (N-terminal) | Linker | CBM (C-terminal) | stability | Linker sequence |
| SEQ ID NO: 394 | SEQ ID NO: | SEQ ID NO: 395 | 0.90 | PPPPPPPPVPPPPG |
| | 422 | | | |
| SEQ ID NO: 394 | SEQ ID NO: | SEQ ID NO: 395 | 0.57 | PPPPPPPPNPPPPG |
| | 423 | | | |
| SEQ ID NO: 394 | SEQ ID NO: | SEQ ID NO: 395 | 0.55 | PPPPPPPPFPPPPG |
| | 424 | | | |
| SEQ ID NO: 394 | SEQ ID NO: | SEQ ID NO: 395 | 0.92 | PPPPPPPPDPPPPG |
| | 425 | | | |

Good linker stability can be obtained when substituting one proline residue with an aromatic, polar, neutral polar or aliphatic, hydrophobic amino acid residue.

### Example 7: Full Scale Washing

The variants have been tested in European Miele front loading wash machine with the conditions specified in Table 5. The variants were tested in wash over 20 repeating wash cycles. Linedrying was used at the end of each test day. Samples of test fabrics, cf. Table 5, were withdrawn after 10, 15 and 20 wash cycles and evaluated by measuring L-values.

Wash conditions are provided in Table 5 below:

TABLE 5
Wash conditions, Full Scale Wash (FSW)

| | Wash conditions |
|---|---|
| Washing machine | Miele EU front loader W1935 |
| Wash program | Cotton wash with following |
| | functions activated; |
| | 1. Short program |
| | 2. Water plus function |
| | Main wash time 51 min |
| Water Volume in main wash | 15 L |
| Water hardness | 15° dH |
| Temperature, ° C. | 40° C. |
| Ballast | 4 kg mixed ballast consisting of |
| | 65% cotton and 35% polyester |
| Test fabric | New EMPA252 |
| | Pre-aged EMPA252 |
| | Test fabric is obtained from Center for |
| | Testmaterials (https://www.cftbv.nl/) |
| Measuring method | L-values from L*, a*, b* |
| | (Equipment: DataColor 800 V |
| | spectrophotometer. L*, a*, b* was |
| | measured under D65 standard light) |
| Detergent | Model detergent B 3.33 g/ L |
| Enzyme | Endoglucanase: 0.05 ppm |
| (mg active enzyme protein pr L | Protease: SEQ ID NO: 406, 1.32 ppm |
| water in main wash) | |

Pre-aged EMPA252 were prepared by washing new EMPA252 in deionized water (no detergent) for 12 hours+12 hours without drying between the two wash cycles. Finally, the swatches were line dried overnight.

Percent relative performance of the endoglucanases is calculated as the delta performance of the variant vs blank (no endoglucanase) divided by the delta performance vs blank of SEQ IN NO: 391, i.e.

$$[ ( {L}_{Blank} - {L}_{Variant} ) / ( {L}_{Blank} - {L}_{SEQ ID NO : 391} ) ] \times 100 \%$$

Data from Examples 3 and 4 show that a clearly improved linker stability can be obtained with the introduction of the proline rich linker PPPPPPPG (SEQ ID NO:378). Table 6 shows that the introduction of the PPPPPPPG linker (SEQ ID NO:378) to increase stability is at the expense of wash performance. The endoglucanase (SEQ ID: NO: 405) with the PPPPPPPG linker (SEQ ID NO: 378) performs significantly inferior to the reference endoglucanase having SEQ ID NO:391, however still significantly superior to the Blank conditions (i.e. without endoglucanase).

TABLE 6
Wash performance data of endoglucanases having SEQ ID NO: 391 and SEQ ID NO: 405,
respectively. Different letters in brackets after performance data indicates that
the results are significantly different when evaluated in SAS JMP by LSMeans
differences Tukey HSD

| Catalytic | | | Wash perfor- | |
|---|---|---|---|---|
| domain | | CBM | mance after | |
| (N-terminal) | Linker | (C-terminal) | 20 FSW cycles | Linker sequence |
| Blank (no endoglucanase) | | | 39.2 (A) | |
| SEQ ID NO: 391 | | | 35.3 (C) | PPSGGNGGTGPTSTAP- |
| | | | | GSGQTSPGGGSG |
| SEQ ID | SEQ ID NO: 378 | SEQ ID | 37.3 (B) | PPPPPPPG |
| NO: 394 | | NO: 395 | | |

Table 7 shows that the problem with inferiority in wash performance of endoglucanase (SEQ ID NO: 405) with the proline rich PPPPPPPG linker (SEQ ID NO:378) can be solved by the introduction of proline rich linkers of the present invention (P/G)ₐG: the wash performance is superior to the wash performance of the endoglucanase (SEQ ID NO:405) with the shorter linker PPPPPPPG (SEQ ID NO:378) and on par with the endoglucanase having SEQ ID NO: 391.

TABLE 7
Performance of endoglucanases relative to reference endoglucanase having SEQ ID
NO: 391

| | | | Wash per- | |
|---|---|---|---|---|
| Catalytic | | | fomance relative | |
| domain | | CBM | to SEQ ID | |
| (N-terminal) | Linker | (C-terminal) | NO: 391 | Linker sequence |
| SEQ ID NO: 391 | | | 100 | PPSGGNGGTGTPTSTA |
| | | | | PGSGQTSPGGGSG |
| SEQ ID NO: 405 | | | 49 | PPPPPPPG |
| SEQ ID | SEQ ID NO: 26 | SEQ ID | 91 | PPPPPGPPPPPPPG |
| NO: 394 | | NO: 395 | | |
| SEQ ID | SEQ ID NO: 97 | SEQ ID | 90 | PPGPPPPPGPPPPG |
| NO: 394 | | NO: 395 | | |
| SEQ ID | SEQ ID NO: 93 | SEQ ID | 96 | PPPPPPPPGPPPPG |
| NO: 394 | | NO: 395 | | |
| SEQ ID | SEQ ID NO: 112 | SEQ ID | 97 | PPGPPGPPGPPPPG |
| NO: 394 | | NO: 395 | | |
| SEQ ID | SEQ ID NO: 4 | SEQ ID | 98 | PPGPPPPPPPPPPG |
| NO: 394 | | NO: 396 | | |

Finally, the endoglucanases with the linker of the form (P/G)ₐG have the same performance as endoglucanase having SEQ ID NO:391 and all perform significantly better than the Blank conditions as can been seen in Table 8, below:

TABLE 8
Different letters in brackets after performance data indicates that the results are
significantly different when evaluated in SAS JMP by LSMeans differences Tukey HSD

| Catalytic | | | Wash perfor- | |
|---|---|---|---|---|
| domain | | CBM | mance after 20 | |
| (N-terminal) | Linker | (C-terminal) | FSW cycles | Linker sequence |
| Blank (no endoglucanase) | | | 39.8 (A) | |
| SEQ ID NO: 391 | | | 35.8 (B) | PPSGGNGGTGTPTSTA |
| | | | | PGSGQTSPGGGSG |
| SEQ ID | SEQ ID NO: 26 | SEQ ID | 36.2 (B) | PPPPPGPPPPPPPG |
| NO: 394 | | NO: 395 | | |
| SEQ ID | SEQ ID NO: 97 | SEQ ID | 36.2 (B) | PPGPPPPPGPPPPG |
| NO: 394 | | NO: 395 | | |
| SEQ ID | SEQ ID NO: 93 | SEQ ID | 36.0 (B) | PPPPPPPPGPPPPG |
| NO: 394 | | NO: 395 | | |
| SEQ ID | SEQ ID NO: 112 | SEQ ID | 36.0 (B) | PPGPPGPPGPPPPG |
| NO: 394 | | NO: 395 | | |
| SEQ ID | SEQ ID NO: 4 | SEQ ID | 35.9 (B) | PPGPPPPPPPPPPG |
| NO: 394 | | NO: 396 | | |

### Example 8: Production and Stability of Co-Granulates

Co-granulates comprising protease (SEQ ID NO: 407) and either commercially available non-stabilized cellulase (SEQ ID NO: 391) or stabilized cellulase of the present invention (SEQ ID NO: 398) were produced according to the following procedure:

Dry ingredients (Na₂SO₄, cellulose fibers, enzyme powder) and wet ingredients (water, sucrose, liquid enzyme concentrate) were mixed in a 50L horizontal high shear mixer, after which the formed wet granules were dried in a fluid bed and thereafter sieved between 250-1200 μm.

Stability of the cellulases in cellulase/protease co-granulates are shown in Table 9, the results show that.
The granulation yield is higher for the stabilized cellulase compared to the non-stabilized cellulase when granulated together with a protease.
The stability under varying storage conditions is much improved for the stabilized cellulase compared to the non-stabilized protease. Mass spectrometry (MS) shows that loss of activity for the non-stabilized cellulase is due to lack of stability of the linker between catalytic domain and CBM.

TABLE 9
Granulation yield and stability with and without protease. All
samples were stored for 2 weeks under the specified conditions.

| | Granulation | Granulation | Stability | | |
|---|---|---|---|---|---|
| | with/out protease | yield | 40° C./60% R.H. | 50° C./closed lids | 40° C./60% R.H. |
| SEQ ID NO: | With protease | 92% | 87% | 95% | 69% |
| 398 | No protease | 86% | 91% | 100% | 67% |
| SEQ ID NO: | With protease | 73% | n.d | n.d | n.d |
| 391 | No protease | 86% | n.d | n.d | n.d |

“n.d” indicates that no intact cellulase was detected.

Additionally, 20 cycle full scale wash of new and pre-aged EMPA 252 textiles with a commercial European powder detergent confirmed that the anti-pilling wash performance of the cellulase having SEQ ID NO: 398 is as good for the cellulase when co-granulated with protease as for the cellulase granulated per se when this is tested with the same amount of protease in the wash as for the co-granulate test—and that the cellulase provides relevant anti-pilling effects compared to the detergent without cellulase. Wash performance results on EMPA 252 textiles were evaluated by L*-vales on black stripes on these textiles.

### Example 9: Stabililty of SEQ ID NO: 398 vs GH45 Cellulase from Acremonium thermophilum

Stability of variant of a GH45 cellulase polypeptide from Acremonium thermophilum (SEQ ID NO: 409 of the present invention) disclosed in EP 3 653 705 was compared with the stability of a cellulase of the present invention having SEQ ID NO: 398 under the following conditions:

0.4 mg/ml of the relevant cellulase and 0.025 mg/mL protease (SEQ ID NO: 408) was dissolved in 20 mM HEPES pH8, 5% Detergent A and incubated under conditions as described in Table 10. Samples were analyzed by SDS-PAGE and Intact mass spectrometry.

TABLE 10
Intact endoglucanase after incubation with protease

| | Intact enzyme after incubation in Model Detergent A | | |
|---|---|---|---|
| | with protease having SEQ ID NO: 408 at 60° C. | | |
| | 10 min | 30 min | 60 min |
| SEQ ID NO: 409 | 0% | 0% | 0% |
| SEQ ID NO: 398 | 98% | 95% | 90% |

Results provided in Table 10 show that the stability of the variant of the present invention is much improved over the commercially available cellulase.

Based on the experimental data outlined in the examples above it becomes evident that stability of the (P/X)ₐG linker is markedly improved while wash performance is maintained when compared to the commercially available endoglucanase having SEQ ID NO: 39. Consequently, it can be concluded that the problem of obtaining a stable as well as flexibel (enabling wash performance) linker has been solved.

Further the data shows that the (P/X)ₐG linker gives rise to higher granulation yield as well as better storage stability in co-granulates.

### Example 10: Stability in Compact Detergent

The endoglucanases having SEQ ID NO: 391 and SEQ ID NO: 398, respectively, are stored in 5 g Detergent C under the conditions and concentrations outlined in Table 11.

TABLE 11
Storage conditions for endoglucanases in compact detergent.

| | Protease | Endoglucanase | Endoglucanase | Protease mg | Storage | |
|---|---|---|---|---|---|---|
| | SEQ ID NO | SEQ ID NO | mg EP/g detergent | EP/g detergent | conditions | |
| 1 | 406 | 398 | 0.2 | 0.040 | −18° | C. |
| 2 | 406 | 398 | 0.2 | 0.040 | 37° | C.@2 W |
| 3 | 406 | 391 | 0.2 | 0.040 | −18° | C. |
| 4 | 406 | 391 | 0.2 | 0.040 | 37° | C.@2 W |

Intact endoglucanase is analysed by mass spectrometry (MS) and confirms that the stability of the endoglucanase variant having SEQ ID NO: 398 is improved over the stability of the endoglucanase variant having SEQ ID NO: 391.

1. A polypeptide having endoglucanase activity, characterized in that said polypeptide consists of a catalytic domain, a linker, and a carbohydrate binding module (CBM), wherein the linker is of the form (P/X) G wherein the value of a is in the range 8-16, X is selected from the amino acids G, V, N, F and D, and (P/X)ₐ specifies that each position in the linker is selected from the group consisting of the amino acids G, V, N, F, D and P, and wherein further the linker comprises at least one and not more than five amino acids selected from the group consisting of G, V, N, F and D.

2. The polypeptide of claim 1, characterized in that the catalytic domain has a least 80% sequence identity to SEQ ID NO:394.

3. The polypeptide of claim 1, characterized in that the CBM has at least 80% sequence identity to SEQ ID NO:395 or to SEQ ID NO:396.

4. The polypeptide of claims 1, wherein each of the G, V, N, F and D when present in the linker are separated by at least one P, such as 1, 2, 3, 4, 5, 6, 7, 8, 9, 10, 11, 12, 13, 14 or 15 P.

5. The polypeptide according to claim 1, wherein the at least the first amino acid residue in the linker, such as the first 1, 2, 3, 4, 5, 6, 7, 8, 9, 10, 11, 12, 13, 14 or 15 amino acid residues, is P.

6. The polypeptide according to claim1, wherein
a. X is a glycine residue (G), so that (P/X)ₐ specifies that each position in the linker is selected from the group of amino acids consisting of P and G,
b. the value of a is in the range 10-16, preferably 13 to 16, preferably 13
c. the linker comprises 2, 3, 4 or 5 G, preferably 2, 3 or 4, preferably 2 or 3 G,
d. each of the glycine residues are separated by at least one P, such as 1, 2, 3, 4, 5, 6, 7, 8, 9, 10, 11, 12, 13 or 14 P, and
e. wherein the linker further has at least one P before the first G in the linker, such 1, 2, 3, 4, 5, 6, 7, 8, 9, 10, 11, 12, 13 or 14 P before the first G in the linker.

7. The polypeptide according to claim 1, wherein the catalytic domain has at least 80% sequence identity to SEQ ID NO:394, such as at least 85%, 90%, 91%, 92%, 93%, 94%, 95%, 96%, 97%, 98%, 99% or even 100% sequence identity to SEQ ID NO:394.

8. The polypeptide according to claim 1, wherein the CBM has at least 91%, such as 92%, 93%, 94%, 95%, 96%, 97%, 98%, 99% or even 100% sequence identity to SEQ ID NO:395 or to SEQ ID NO:396.

9. The polypeptide according to claim 1, wherein the linker is selected from the group consisting of SEQ ID NO:26, SEQ ID NO:97, SEQ ID NO:93 and SEQ ID NO: 404.

10. The polypeptide according to claim 1, wherein the polypeptide is selected from the group of polypeptides having at least 80% sequence identity, such as 85%, 90%, 91%, 92%, 93%, 94%, 95%, 96%, 97%, 98%, 99% or even 100% sequence identity to any of SEQ ID NO: 397, SEQ ID NO:398, SEQ ID NO: 399, SEQ ID NO: 400, and SEQ ID NO:401.

11. The polypeptide according to claim 1, wherein the linker stability in an aqueous composition comprising a protease as determined by the Linker Stabililtiy Assay (Example 2) is at least 0.1, or 0.15 or 0.2.

12. A composition comprising one or more of the polypeptides of claim 1, at least one detergent component and optionally one or more additional enzymes such as protease.

13. (canceled)

14. A polynucleotide encoding the polypeptide of claim 1.

15. A nucleic acid construct or expression vector comprising the polynucleotide of claim 14.

16. A recombinant host cell transformed with the polynucleotide of claim 14.

17. A method of producing a polypeptide, comprising:
a. cultivating the recombinant host cell of claim 16 under conditions suitable for expression of the variant; and
b. recovering the variant.

18. A method for cleaning a surface, such as a textile, comprising contacting the surface with a polypeptide of claim 1.

19. The composition according to claim 12, further comprising a protease.

20. The composition of claim 19, wherein the protease is formulated as a co-granulate.

21. A method for cleaning a surface, such as a textile, comprising contacting the surface with a composition of claim 12.
